(19) United States
(12) Patent Application Publication
(10) Pub. No.: US 2013/0012404 A1
(43) Pub. Date: Jan. 10, 2013

(54) CULTURE METHOD, EVALUATION METHOD AND STORAGE METHOD FOR CANCER-TISSUE-DERIVED CELL MASS OR AGGREGATED CANCER CELL MASS

(75) Inventor: Masahiro Inoue, Osaka (JP)

(73) Assignees: Osaka Prefectural Hospital Organization; Renaissance Energy Investment Co., Ltd.

(21) Appl. No.: 13/522,877

(22) PCT Filed: Jan. 19, 2011
(86) PCT No.: PCT/JP2011/050866

§ 371 Date: Sep. 26, 2012

(30) Foreign Application Priority Data
Jan. 19, 2010 (JP) 2010-009292

(51) Int. Cl. C12N 5/09; C12Q 1/68; C40B 30/04; C12Q 1/04

(52) U.S. Cl. 506/9; 435/325; 435/34; 435/6.11; 435/6.12; 435/374

(57) ABSTRACT

It is intended to provide a method for culturing a novel cancer tissue-derived cell mass or a novel aggregated cancer cell mass that can reflect the behavior of cancer cells accurately in vivo. First, a cancer tissue-derived cell mass or an aggregated cancer cell mass is prepared from an individual. The novel cancer tissue-derived cell mass or the novel aggregated cancer cell mass is cultured, and the properties are evaluated using the cultured cell mass. Examples of the evaluation of properties include the evaluation of genes and the evaluation of culture conditions. In addition, the cancer tissue-derived cell mass or the aggregated cancer cell mass can be stored. It is possible to establish an optimal therapeutic method for an individual efficiently by linking the clinical information or the genetic information on the individual to the stored cancer tissue-derived cell mass or the stored aggregated cancer cell mass.

### TECHNICAL FIELD

The present invention relates to a culture method, an evaluation method and a storage method for a cancer tissue-derived cell mass or an aggregated cancer cell mass. More particularly, the present invention relates to a culture method, an evaluation method and a storage method for a cancer tissue-derived cell mass or an aggregated cancer cell mass that can reconstruct a cancer in vitro and retain the proliferation ability.

### BACKGROUND ART

In recent years, therapeutic results of early-stage cancers have been drastically improved as a result of various studies that have been repeated to overcome cancers. However, it is still difficult to treat advanced-stage cancers, and cancers have continued to occupy the first place of the Japanese cause of death. According to vital statistics of 2007 by the Ministry of Health, Labour and Welfare, 340,000 people or more died of cancers a year.

For cancer research so far, especially when examining its behavior in vitro, experiments using a cancer cell line that has been subcultured and established under optimized culture conditions are the mainstream. These cancer cell lines include human breast cancer cell lines (MDF7, NCI/ADR HS578T, MDA-MB-22231/ATCC, MDA-MB-4335, MDA-N, BT-549, T-47D), human cervical cancer cell lines (HeLa), human lung cancer cell lines (A549, EKVX, HOP-62, HOP-92, NCI-H23, NCI-H226, NCI-H322M, NCI-H460, NCI-H522), human colon cancer cell lines (Caco-2, COLO 205, HCC-2998, HCT-15, HCT-116, HT29, KM12, SW-620), and human prostate cancer cell lines (DU-145, PC-3, LNCaP), etc., which have been practically widely used for research.

For a realization of diagnosis or treatment according to cancer patients, it is said that primary culture of cancer cells is promising, and its research has been advanced. For example, a CD-DST method (Collagen gel droplet embedded drug sensitivity test) using a primary culture cell has been developed. This in vitro test method is a drug sensitivity test by embedding a tissue or a cell isolated from a patient into a collagen gel droplet, and examining the sensitivity by the combination of a three-dimensional culture and an image colorimetric quantification (for example, Non-Patent Document 1). However, as to the primary culture cell, its culture method has not been established yet, and its handling is difficult.

As a result of studies on cancer cells, cancer cells constituting a cancer may consist of a plurality of subpopulations which are each a small population called as “tumor initiating cells” or “tumor stem cells” able to self-replicate, and a series of reports that support the existence of such subpopulations which are able to become a source of the majority of cancer cells through differentiation have been published (for example, Non-Patent Documents 2 and 3). Such stem cells can be obtained, for example, by separating a tumor removed from a living body into single cells and sorting them. Some of them are said to have a proliferation ability even in vitro (Non-Patent Document 4). However, there is a negative report (Non-Patent Document 5) to the theory to explain the origin of cancer in terms of the stem cell in this way, and thus such a theory still remains a hypothesis.

There are still many unknown points about cancer even in the current state where cancer research has been widely performed.

### PRIOR ART DOCUMENTS

### Non-Patent Documents

Non-Patent Document 1: Takamura et al., (2002) Prediction of chemotherapeutic response by collagen gel droplet embedded culture-drug sensitivity test in human breast cancers. Int. J. Cancer, 98, 450-455
Non-Patent Document 2: Vermeulen L, at al., (2008) Single-cell cloning of colon cancer stem cells reveals a multi-lineage differentiation capacity. PNAS Vol. 105 No. 36 13427-13432
Non-Patent Document 3: Ricci-Vitiani L, at al., (2007) Identification and expansion of human colon-cancer-inhibiting cells. Nature Vol. 445 111-115
Non-Patent Document 4: Todaro M, et al., (2007) Colon cancer stem cells dictate tumor growth and resist cell death by production of interleukin-4. Cell Stem Cell 1:389-402
Non-Patent Document 5: Shmelkov S V, et al., (2008) CD133 expression is not restricted to stem cells, and both CD133+ and CD133− metastatic colon cancer cells initiate tumors. The Journal of Clinical Investigation Vol. 118 2111-2120

### SUMMARY OF THE INVENTION

### Problems to be Solved by the Invention

An object of the present invention is to provide a culture method, a hormone dependency or gene evaluation method, and a storage method for a novel cancer tissue-derived cell mass or a novel aggregated cancer cell mass that can reproduce in vitro the behavior of in vivo cancer cells, can accurately verify the state of in vivo cancer cells, and is useful as a sample for the study of analysis and treatment of cancer.

### Means for Solving the Problems

The present inventor has aimed to test a therapeutic sensitivity for individual cancer patients taking into consideration that there are many problems to be solved, such as a possible difference in nature of cell lines having been used as study materials for cancer research, from patient's cancers, and a low cell survival rate in a miscellaneous cell population of primary culture cells. As a result of diligent studies on culturing of cancer cells as research materials in order to solve the above problems, the present inventor has prepared a novel cancer tissue-derived cell mass or a novel aggregated cancer cell mass and found that such a cell mass can be cultured, stored, and used for various evaluations. The present invention has been completed based on these findings.

In other words, an object of the present invention is to provide a novel culture method, a novel storage method, and novel various evaluation methods for a novel cancer tissue-derived cell mass or a novel aggregated cancer cell mass that can accurately reflect in vitro the behavior of in vivo cancer cells in individuals.

The present invention is a method for culturing a cancer tissue-derived cell mass or an aggregated cancer cell mass, comprising culturing the cancer tissue-derived cell mass or the aggregated cancer cell mass in a culture medium obtained by adding a serum replacement to a serum-free basal culture medium.

The culture medium obtained by adding a serum replacement to a serum-free basal culture medium may be STEMPRO (registered trademark).

The cancer tissue-derived cell mass or the aggregated cancer cell mass may be derived from colorectal cancer, ovarian cancer, breast cancer, lung cancer, prostate cancer, uterine cancer, kidney cancer, bladder cancer, pharyngeal cancer, or pancreatic cancer.

In addition, the culture may be carried out with the addition of a hormone to the culture medium.

The cancer tissue-derived cell mass or the aggregated cancer cell mass may be derived from a cancer selected from the group consisting of breast cancer, uterine cancer, and prostate cancer, and the hormone may be at least a hormone selected from the group consisting of estrogen, progesterone, and testosterone.

The cancer tissue-derived cell mass or the aggregated cancer cell mass may be treated to divide every fixed period of time during the culture.

In addition, the present invention relates to a method for evaluating hormone dependency of a cancer tissue-derived cell mass or an aggregated cancer cell mass, comprising the steps of culturing the cancer tissue-derived cell mass or the aggregated cancer cell mass in the presence or absence of a hormone; and comparing the state of the cancer tissue-derived cell mass or the aggregated cancer cell mass by the presence or absence of the hormone after the culture.

The cancer tissue-derived cell mass or the aggregated cancer cell mass may be derived from a cancer selected from the group consisting of breast cancer, uterine cancer, and prostate cancer, and the hormone may be at least a hormone selected from the group consisting of estrogen, progesterone, and testosterone.

The comparison step may be to compare the state of proliferation or the state of life and death of the cancer tissue-derived cell mass or the aggregated cancer cell mass.

The present invention also relates to a method for evaluating a cancer tissue-derived cell mass or an aggregated cancer cell mass, comprising the steps of culturing the cancer tissue-derived cell mass or the aggregated cancer cell mass; and evaluating the gene of the cultured cancer tissue-derived cell mass or the cultured aggregated cancer cell mass.

The gene may be a KRAS gene or a BRAF gene, and the evaluation may be to detect the presence or absence of a gene mutation.

The step of evaluating the gene may be to detect the expression level of the gene.

The culturing may be carried out in a hypoxic state or in a normal oxygen state and the step of evaluating the gene may be to compare the expression level of the gene in the culture in the hypoxic state or in the normal oxygen state.

The gene may be a VEGF gene.

The present invention also relates to a method for storing a cancer tissue-derived cell mass or an aggregated cancer cell mass by a freezing method.

The storage method may be a method comprising a unicellularization treatment of a cancer tissue-derived cell mass and a treatment for promoting cell aggregation or drug treatment for suppressing cell death.

The unicellularization treatment may be a treatment using one kind selected from the group consisting of trypsin, dyspase, and optionally collagenase, papain, hyaluronidase, C. histolyticum neutral protease, thermolysin, and dispase, or a combination of two or more enzymes thereof, and the treatment for promoting cell aggregation or the drug treatment for suppressing cell death may a treatment with a ROCK inhibitor or a caspase inhibitor.

The storage method may be carried out by a vitrification method.

The cancer tissue-derived cell mass or the aggregated cancer cell mass may be stored in a state associated with genetic information belonging to the cancer tissue-derived cell mass or the aggregated cancer cell mass.

The cancer tissue-derived cell mass or the aggregated cancer cell mass may be stored in a state associated with clinical information derived from a patient.

The cancer tissue-derived cell mass or the aggregated cancer cell mass may be stored in a state associated with information of culture conditions for the cancer tissue-derived cell mass or the aggregated cancer cell mass.

The information of culture conditions may be the presence or absence of hormone dependency.

### Effect of the Invention

The cancer tissue-derived cell mass or the aggregated cancer cell mass according to the present invention can be cultured over a long period of time while retaining its proliferation ability by adjusting the culture conditions. In addition, it is possible to store the cell mass and associate the cell mass with genetic information or clinical information. In this way, it becomes possible to establish quickly and accurately an optimal therapeutic method that is not uniform and corresponds to individual patients.

### BRIEF DESCRIPTION OF THE DRAWINGS

FIG. 1 is a drawing showing the formation process of the cell mass derived from a cancer tissue according to the present invention.

FIG. 2 shows an embodiment of the cell mass derived from a cancer tissue according to the present invention, wherein the cell expresses a surface antigen such as CD133, CD44, CD166, etc, respectively from the left.

FIG. 3 is a drawing showing the change of form and proliferation ability during the in vitro culture process of the cell mass derived from a cancer tissue according to the present invention. The status at the day 0, 13, and 23 from the left in the upper column and day 31 in the bottom column are shown.

FIG. 4 is a drawing showing the result of an in vitro drug-sensitivity test with 5-FU using the cell mass derived from a cancer tissue according to the present invention.

FIG. 5 is a drawing wherein a tumor tissue (right) obtained by transplanting the cell mass derived from a cancer tissue according to the present invention into mice is compared with a tumor tissue (left) that is removed from a living body from which the cell mass derived from a cancer tissue is derived.

FIG. 6 is a drawing showing the result of an in vitro radiosensitivity test using the cell mass derived from a cancer tissue according to the present invention.

FIG. 7 is a drawing showing the cell mass derived from a cancer tissue according to the present invention, wherein the cell mass is obtained from various cancer tissues, and wherein colon cancer, pancreatic cancer, and ovarian cancer (upper part); pharyngeal cancer, breast cancer, and lung cancer (middle part); and prostate cancer, kidney cancer, and bladder cancer (lower part) are shown from the left.

FIG. 8 is a drawing showing the result of a culture test for hormone sensitivity using the cell mass derived from a breast cancer tissue. The left shows estradiol (−) and the right shows estradiol (+). Each shows the changes from the day 0 to day 6.

FIG. 9 is a drawing showing the cell mass derived from a cancer tissue according to the present invention, wherein the cell mass is obtained from a mouse pancreatic islet cell tumor.

FIG. 10 is a drawing showing the result of comparing the states between before and after cryopreservation of the cell mass derived from a cancer tissue according to the present invention (left: before cryopreservation; right: 24 hours after thawing).

FIG. 11 illustrates a view showing an aggregated cancer cell mass derived from a cancer tissue-derived cell mass.

FIG. 12 illustrates a view showing an aggregated cancer cell mass derived from surgical specimens of human colorectal cancer.

FIG. 13 illustrates views showing the state of an aggregated cancer cell mass that is treated with trypsin, cryopreserved, and then thawed, wherein the left view is on day 0 and the right view is on day 1.

FIG. 14 illustrates a view showing the results of in vitro drug sensitivity test with doxorubicin using an aggregated cancer cell mass.

FIG. 15 illustrates a view showing the detection of gene mutation of KRAS or BRAF by a cancer tissue-derived cell mass.

FIG. 16 illustrates a view showing the results of a test for VEGF expression in a cancer tissue-derived cell mass induced by a normal atmospheric condition and a hypoxic condition.

### MODE FOR CARRYING OUT THE INVENTION

The cell mass derived from a cancer tissue according to the present invention is an isolated product that is isolated from a cancer tissue obtained from an individual as amass containing at least three cancer cells or a culture of the isolated product and which can retain a proliferation ability in vitro.

Here, the expression of “an isolated product that is isolated from a cancer tissue obtained from an individual as amass containing at least three cancer cells” means an isolated product obtained by treatment of a cancer tissue of a cancer that has occurred in a living body and containing at least three cancer cells, preferably at least eight cancer cells. Such an isolated product does not include a product isolated to single cells as well as does not include a composition that has been once separated to single cells and has been then reconstructed. However, this isolated product includes not only a product obtained just after isolation from a living body, but also a product that is kept in, for example, a physiological saline solution for a certain period of time, or a product after freezing or cryopreservation.

The “cancer tissue obtained” from an individual refers to a cancer tissue obtained by surgical removal, etc., as well as a cancer tissue obtained with a needle or an endoscope so that it is possible to handle it in vitro for a tissue examination.

The expression of “a culture of an isolated product that is isolated from a cancer tissue obtained by isolation from an individual as a mass containing at least three cancer cells” refers to a product obtained by culturing in vitro an isolated product obtained by isolation from a cancer tissue of a cancer that has occurred in a living body as a mass containing at least three cancer cells. The culture time is not particularly limited, and the culture may include a culture that is allowed to be present in a medium even for a short time. This culture often takes an almost spherical or ellipsoidal form after being cultured for a certain period of time, preferably for at least three hours. The culture as described herein includes not only a culture with an almost spherical or ellipsoidal form after such a certain period of time, but also a culture with an irregular form before reaching such a spherical or ellipsoidal form. In addition, the culture as described herein includes a culture with an irregular form obtained by dividing such an almost spherical or ellipsoidal form, and a culture having an almost spherical or ellipsoidal form after further culture.

The expression of “can retain a proliferation ability” means that the cell mass derived from a cancer tissue according to the present invention can retain a proliferation ability in vitro for at least 10 days, preferably at least 13 days, and more preferably at least 30 days, under cell culture conditions of a temperature of 37° C. and a 5% CO₂-incubator.

Although such a cell mass derived from a cancer tissue can retain a proliferation ability while continuing to culture without mechanical division for a period of at least 10 days, preferably at least 13 days, and more preferably at least 30 days, the proliferation ability can be retained substantially indefinitely by mechanically dividing the cell mass periodically during the culture.

The mechanical division of the cell mass can be performed using a surgical scalpel, knife, scissors, as well as an ophthalmic pointed knife. Alternatively, the mechanical division can also be performed by attaching an injection needle to a syringe and repeating suction and discharge of the cell mass derived from a cancer tissue together with a culture fluid. For example, a 1 ml syringe and a 27 G needle are, but not limited to, preferably used in the present invention.

Here, the medium for culture of the cell mass derived from a cancer tissue according to the present invention is not particularly limited, but an animal cell culture medium is preferably used. Especially preferably, a serum-free medium for stem cell culture is used. Such a serum-free medium is not limited at all so long as it can be used for stem cell culture. The serum-free medium refers to a medium which does not contain a non-adjustable and non-purified serum, and it can be used after addition of a purified blood-derived component or an animal tissue-derived component (e.g., a growth factor).

The serum-free medium of the present invention can be prepared using a medium used for animal cell culture as a basal medium. The basal medium includes, for example, BME medium, BGJb medium, CMRL 1066 medium, Glasgow MEM medium, Improved MEM Zinc Option medium, IMDM medium, Medium 199 medium, Eagle MEM medium, αMEM medium, DMEM medium, RPMI 1640 medium, Fischer's medium, and a combination thereof.

It is possible to culture the cell mass derived from a cancer tissue of the present invention by adding a serum substitute to such a serum-free medium. The serum substitute may be those appropriately containing, for example, albumin, an amino acid (e.g., non-essential amino acids), transferrin, a fatty acid, insulin, a collagen precursor, a trace element, 2-mercaptoethanol or 3′-thiolglycerol, or an equivalent thereof.

In the culture method of the present invention, a commercially available serum substitute can also be used. Examples of such a commercially available serum substitute include a knockout serum replacement (KSR), a Chemically-defined Lipid concentrated (manufactured by Gibco Company), and a Glutamax (manufactured by Gibco Company).

The medium used for culturing the cell mass derived from a cancer tissue according to the present invention can also contain vitamins, growth factors, cytokines, antioxidants, pyruvic acid, buffers, inorganic salts, etc.

In particular, any serum-free media, such as a serum-free medium containing EGF and bFGF, for example, a serum-free medium containing a serum substitute [e.g. knockout serum replacement (KSR, manufactured by Invitrogen Corporation)] and bFGF can be preferably used. The content of the serum substitute or EGF is preferably 10 to 30% w/v based on the whole medium.

Such a medium is not limited, but a commercially available product includes a STEMPRO serum-free medium (Gibco) for human ES cells.

A culture vessel used for culturing the cell mass derived from a cancer tissue can include, but not particularly limited to, for example, flask, flask for tissue culture, dish, petri dish, dish for tissue culture, multi dish, micro plate, micro-well plate, multi plate, multi-well plate, chamber slide, schale, tube, tray, culture bag, and roller bottle, as long as the vessel is generally capable of culturing an animal cell therein.

The culture vessel can be cellular non-adhesive, and a three-dimensional culture is preferably performed in a medium in which a cell supporting substrate (e.g. an extracellular matrix (ECM), etc.) should be co-present. The cell supporting substrate can be any material intended to attach the cell mass derived from a cancer tissue. Examples of such a cell supporting substrate include Matrigel using an extracellular matrix, such as collagen gel, gelatin, poly-L-lysine, poly-D-lysine, laminin, fibronectin, etc. These conditions are preferably used particularly for the proliferation of the cell mass derived from a cancer tissue according to the present invention.

Other culture conditions can be appropriately set. For example, the culture temperature can be, but not limited to, about 30 to 40° C. and most preferably 37° C. The CO₂ concentration can be, for example, about 1 to 10% and preferably about 2 to 5%.

The cell mass derived from a cancer tissue according to the present invention can be cultured in such a medium under such a culture condition. Furthermore, for the culture of the cell mass derived from a cancer tissue, coculture with other cells may be desirable in some cases depending on individual properties, or a special additional supplement such as hormones may be necessary in some cases.

Specifically, coculture may be performed in the presence of feeder cells. For the feeder cells, stromal cell and the like such as fetal fibroblast may be used. Specifically, NIH3T3 and the like are preferable, but not limited to them.

Alternatively, in the case of a specific kind of breast cancer, uterine cancer, and prostate cancer, culture of such a cancer mass is performed preferably in the presence of a hormone. Specifically the hormone includes, but not limited to, estrogen for breast cancer, progesterone for uterine cancer, and testosterone for prostate cancer, and culture conditions can be conveniently adjusted while adding various hormones. In addition, hormone dependence of a cancer derived from a patent is understood by examining how behavior after culture of the cell mass derived from a cancer tissue is changed in the presence of such a hormone. As a result, effectiveness of an anti-hormone therapy may be predicted.

It is also possible to culture the cell mass derived from a cancer tissue according to the present invention by suspension culture. In the floating culture, the cell mass derived from a cancer tissue is cultured in a medium under a non-adhesive-condition to a culture vessel. Such a floating culture includes an embryoid culture method (see Keller et al., Curr. Opin. Cell Biol. 7, 862-869 (1995)), and an SFEB method (for example, Watanabe et al., Nature Neuroscience 8, 288-296 (2005); International Publication No. WO 2005/123902). The floating culture may be used in the production and maintenance of a stable cell mass derived from a tissue culture, which cell mass has, but not particularly limited to, an almost spherical form and has a basement membrane in some cases.

The cell mass derived from a cancer tissue according to the present invention includes a product just after isolation from the cell mass derived from a cancer tissue of an individual, a product after freezing or cryopreservation, and further a cultured product thereof. The culture may be carried out for a period of time, such as preferably for three hours or more, more preferably for 10 hours or more, still more preferably, for 24 hours or more. The culture may be carried out for more than those hours. The cultured product shows specific form such as sphere or the like.

The cancer cells constituting a cell mass derived from a cancer tissue is composed of at least three cancer cells, preferably at least eight cancer cells, more preferably at least ten cancer cells, still more preferably at least 20 cancer cells, and most preferably at least 50 cancer cells. In the case where the cell mass derived from a cancer tissue according to the present invention is an isolated product, it includes preferably 1000 cancer cells or less, and more preferably about 500 cancer cells or less. In the case of a culture after culturing the isolated product, it is possible to increase the number of the cancer cells by culture. However, even the culture contains preferably 10,000 cancer cells or less, and more preferably 5000 cancer cells or less.

The term of “cancer cell” as used in the present invention is used in the sense commonly used, and refers to a cell where an order to be seen in normal cells is disordered, such as unrestricted division/proliferation and escape from apoptosis in a living body. More particularly, the term refers to a cell which has lost a control function for cell proliferation or refers to an extremely attenuated cell, and a cell which has typically acquired an infinite proliferation ability at high frequency of 80% or more, many of which also have an ability of invasion and metastasis, and, as a result, are regarded as a malignant neoplasm that causes the death particularly in a mammal including a human.

In the present invention, the kind of the tissue derived from a cancer is not particularly limited, but it can be derived from cancers that occur in an animal including a mammal, such as a lymphoma, a blastoma, a sarcoma, a liposarcoma, a neuroendocrine tumor, a mesothelioma, a neurinoma, a meningioma, an adenoma, a melanoma, a leukemia, and a malignant lymphoma, etc., and particularly preferably a carcinoma that occurs in mammalian epithelial cells. Examples of such a carcinoma that occurs in mammalian epithelial cells include a non-small cell lung cancer, a hepatocyte cancer, a bile duct cancer, an esophagus cancer, a stomach cancer, a colorectal cancer, a pancreatic cancer, a cervical cancer, an ovarian cancer, an endometrial cancer, a bladder cancer, a pharyngeal cancer, a breast cancer, a salivary gland cancer, a kidney cancer, a prostate cancer, a labia cancer, an anal cancer, a penis cancer, a testicular cancer, a thyroid cancer, and a head and neck cancer. The animal including a mammal includes, but not particularly limited to, an animal belonging to Primates such as monkey and human, an animal belonging to Rodentia such as mouse, squirrel, and rat, an animal belonging to Lagomorphahe, and an animal belonging to Carnivora such as dog and cat.

Among them, the cell mass of the present invention is particularly preferably derived from, but not limited to, a colon cancer tissue, an ovarian cancer tissue, a breast cancer tissue, a lung cancer tissue, a prostate cancer tissue, a kidney cancer tissue, a bladder cancer tissue, a pharyngeal cancer tissue, or especially a pancreatic cancer tissue.

In the case of a cell mass derived from a cancer tissue such as a colon cancer tissue, the cancer cell contained therein is not particularly limited, but may express CD133.

Isolation of the cancer tissue obtained from a cancer that occurs in a living body is not limited, but includes an enzymatic treatment of a cancer tissue obtained from an individual.

The enzymatic treatment can be a treatment using one member of enzymes selected from collagenase, trypsin, papain, hyaluronidase, C. histolyticum neutral protease, thermolysin, and dispase, or a combination of two or more enzymes thereof. The conditions for such an enzymatic treatment may be as follows: in an isotonic salt solution (e.g. PBS or Hanks' balanced salt solution) buffered at a physiologically acceptable pH (e.g. about pH 6 to 8, preferably about pH 7.2 to 7.6) at for example about 20 to 40° C., preferably at about 25 to 39° C., for a time sufficient to degrade a connective tissue, for example, for about 1 to 180 minutes, preferably 30 to 150 minutes, with a sufficient concentration for such degradation, for example, about 0.0001 to 5% w/v, preferably about 0.001% to 0.5% w/v.

The conditions for such an enzymatic treatment include, but not limited to, a treatment with a mixed enzyme containing collagenase. For example, the enzymatic treatment includes a treatment with a mixed enzyme comprising one or more proteases selected from the group consisting of C. histolyticum neutral protease, thermolysin, and dispase, and one or more collagenases selected from the group consisting of collagenase I, collagenase II, and collagenase IV.

Such a mixed enzyme is not limited, but includes LIBERASE BLENDZYME 1 (registered trade mark) and the like.

The cell mass derived from a cancer tissue according to the present invention comprising optionally a population of at least three cancer cells may take an almost spherical or ellipsoidal form.

The cell mass may contain, but not limited to, a basement membrane-like material present in the circumference of said cancer cell population.

Here, the cancer cells constituting a population often have one or more surface antigens selected from the group consisting of, but not particularly limited to, CD133, CD44, CD166, CD117, CD24 and ESA on the cell surface. CD133, CD44, CD166, CD117, CD24 and ESA are surface antigens that are generally expressed in the cells such as leucocytes (e.g. lymphocytes), fibroblasts, epithelial cells, and cancer cells. These surface antigens are involved in various signal transmission in addition to a function of cell-cell adhesion and cell-matrix adhesion, and can also be surface markers for various stem cells.

When cell groups “express” surface antigens such as CD133 in the present invention, the term “express” means a state where 80% or more of the cells present in the cell groups, preferably 90% or more of the cells present in the cell groups, and more preferably a substantially whole of the cells present in the cell groups represent surface antigens.

In the present specification, the term “basement membrane-like material” refers, but not limited to, a substance that contains preferably at least one member selected from proteoglycans, such as collagen, laminin, nidogen and heparan sulfate proteoglycan; and glycoproteins, such as fibronectin. In the present invention, a basement membrane-like material containing laminin is preferable.

Laminin is a high molecular weight glycoprotein that constitutes a basement membrane. The function of the laminin extends to a wide range, and is involved in, for example, cell functions such as cell adhesion, intercellular signal transmission, and proliferation of normal cells and cancer cells. The laminin has a structure wherein three different subunits are bonded to each other through a disulfide bond, and 11 kinds of laminins have been found depending on the different kinds of each subunit.

Of these, laminin-5 is usually produced only from an epithelial cell, and it is known as a component having activities to adhere to the basement membrane of the epithelial cell and promote a motor function. This laminin-5 has a composite structure that is formed from each one of α3 chain, β3 chain, and γ2 chain, and it is thought that particularly the γ2 chain is inherent to LN5 and is not contained in other LN molecular species.

The cell mass derived from a cancer tissue according to the present invention may have a configuration such that the outer circumference of a population of cancer cells is, as a whole, wrapped in a film which is formed by such a basement membrane-like material. Such a form can be analyzed by observation of the cell mass derived from a cancer tissue with an electron microscope, or by immunostaining of a basement membrane component, or by a combination thereof.

The presence of laminin can be detected, for example, by contacting an antibody that recognizes laminin (e.g. a rabbit antibody derived from a mouse laminin; Sigma-Aldrich Corporation) with a cell mass derived from a cancer tissue, and measuring the antigen-antibody reaction.

Moreover, it is also possible to use a specific antibody that can specify even the kind of the laminin. For example, the presence of laminin-5 can be detected, for example, by contacting an antibody that is reactive particularly to the above inherent γ2-chain or its fragment, with a cell mass derived from a cancer tissue, and measuring the reaction with the antibody.

In the cell mass derived from a cancer tissue according to the present invention, it is desirable that a thin filmy basement membrane-like material is formed in a size of about several micrometers, or about 40 to 120 nm, according to the size of masses, but the size is not limited to them.

The size of the cell mass derived from a cancer tissue according to the present invention also includes, but not limited to, an irregular form with a particle size or a volume average particle size of about 8 μm to 10 μm, as well as further includes a particle size of 1 mm or more of the cell mass that has been grown up greatly after incubation. The diameter of the cell mass is preferably 40 μm to 1000 μm, more preferably 40 μm to 250 and further more preferably 80 μm to 200 μm.

The cell mass derived from a cancer tissue according to the present invention often has one or more arrangements particularly selected from the group consisting of, but not particularly limited to, palisade arrangement, sheet arrangement, multilayer arrangement, and syncytial arrangement.

The cell mass derived from a cancer tissue according to the present invention may be prepared typically by a process which comprises the steps of treating a fragmented product of a cancer tissue removed from a living body, with an enzyme; and selecting and collecting a mass containing at least three cancer cells among from an enzymatic treatment product.

Moreover, the cell mass derived from a cancer tissue according to the present invention may be prepared by, but not limited to, a process comprising the step of culturing the thus collected component for three or more hours.

At first the cancer tissue removed from a living body can be fragmented as it is, or the cancer tissue is first maintained in a medium for animal cell culture before fragmentation. The medium for animal cell culture includes, but not particularly limited to, Dulbecco's MEM (DMEM F12, etc.), Eagle's MEM, RPMI, Ham's F12, alpha MEM, and Iscove's modified Dulbecco's medium. In this case, floating culture is preferably carried out in a culture vessel which is non-cell-adhesive.

It is also preferable to wash the cancer tissue in advance for fragmentation. Such a washing can be carried out using, but not limited to, a buffer solution such as acetic acid buffer solution (acetic acid+sodium acetate), phosphoric acid buffer solution (phosphoric acid+sodium phosphate), citric acid buffer solution (citric acid+sodium citrate), boric acid buffer solution, tartaric acid buffer solution, Tris buffer solution, and phosphate-buffered saline. In the present invention, washing of the tissue can be performed particularly preferably in HBSS. As for the number of times of the washing, once to three times are suitable.

The fragmentation can be performed by dividing the tissue after washing, with use of a knife, scissors, or a cutter (manual operation and automatic operation). The size and form after fragmentation are not particularly limited, but the fragmentation may be performed at random. The tissue is fragmented to a uniform size, preferably 1 mm to 5 mm square, more preferably 1 mm to 2 mm square.

The fragmented product thus obtained is then subjected to an enzymatic treatment. Such an enzymatic treatment can be a treatment using one member of enzymes selected from collagenase, trypsin, papain, hyaluronidase, C. histolyticum neutral protease, thermolysin, and dispase, or a combination of two or more enzymes thereof. The conditions for such an enzymatic treatment may be as follows: in an isotonic salt solution (e.g. PBS or Hank's balanced salt solution) buffered at a physiologically acceptable pH (e.g. about pH 6 to 8, preferably about pH 7.2 to 7.6) at for example about 20 to 40° C., preferably at about 25 to 39° C., for a time sufficient to degrade a connective tissue, for example, about 1 to 180 minutes, preferably about 30 to 150 minutes, with a sufficient concentration for such degradation, for example, about 0.0001 to 5% w/v, preferably about 0.001% to 0.5% w/v.

The conditions for this enzymatic treatment include, but not limited to, a treatment using a mixed enzyme containing, for example, collagenase. More preferably, the enzymatic treatment includes a treatment with a mixed enzyme comprising at least one protease selected from the group consisting of C. histolyticum neutral protease, thermolysin, and dispase, and at least one collagenase selected from the group consisting of collagenase I, collagenase II, and collagenase IV.

Such a mixed enzyme includes, but not limited to, LIBERASE BLENDZYME 1 (registered trade mark) and the like.

Among the enzymatic treatment products obtained in this way, it is preferable to select and collect a mass containing at least three cancer cells. The process for such selection and collection is not particularly limited, but any process well-known to those skilled in the art for assorting the size can be used.

Of the methods for assorting the size, a simple and easy process is a visual observation, a classification with a phase contrast microscope, or a classification with a sieve, but the classification method is not particularly limited so long as it is a classification with a particle size available for those skilled in the art. When a sieve is used, it is preferable to collect a component which passes through a sieve with a mesh size of 20 μm and does not pass through a sieve with a mesh size of 500 μm. It is more preferable to collect a component which passes through a sieve with a mesh size of 40 μm and does not pass through a sieve with a mesh size of 250 μm.

Here, the mass containing at least three cancer cells, which is a subject for selection, is a cell mass derived from a cancer tissue according to the present invention and has a certain range of sizes. The term of “a certain range of sizes” includes small ones with a volume average particle size of about 8 μm to 10 μm. When the cell mass is in an almost sphere form, it has a diameter of 20 to 500 μm, preferably 30 to 400 μm, and more preferably 40 to 250 μm. When the cell mass is in an ellipsoidal form, it has a long diameter of 20 to 500 μm, preferably 30 to 400 μm, and more preferably 40 to 250 μm. When the cell mass is in an irregular form, it has a volume average particle size of 20 to 500 μm, preferably 30 to 400 μm, and more preferably 40 to 250 μm. The measurement of the volume average particle size can be performed by evaluating a particle size distribution and a particle shape using a CCD camera attached to a phase contrast microscope (IX70; manufactured by Olympus Corporation).

Both of the isolated product and its culture product, which are components obtained in this way by selection and collection, are a cell mass derived from a cancer tissue according to the present invention. The culture product may be those wherein the isolated product as a component after selection and collection has been present in a medium for a short time, or those which are in an almost sphere or ellipsoidal form after culture for a period of time, for example, at least three hours, preferably 10 to 36 hours, and more preferably 24 to 36 hours or more. The culture time may be over 36 hours, several days, at least 10 days, at least 13 days, or at least 30 days.

The culture may be performed in a medium for a long time without any mechanical division, but a proliferation ability can also be retained for a substantially infinite time period by a mechanical division periodically on the way of culture.

The cancer tissue-derived cell mass of the present invention, even if it includes, for example, 10 or less cancer tissue-derived cell masses (equivalent to 1000 or less cells) with a diameter of 100 μm, has a high engraftment rate in the transplantation in different species of animal. Therefore, the cancer tissue-derived cell mass of the present invention is useful in the simple and easy production of a cancer model animal including a mouse, and makes it possible to examine a cancer tissue more strictly, evaluate drug sensitivity, or evaluate therapeutic embodiments including a radiation therapy.

It is possible to cryopreserve the cancer tissue-derived cell mass of the present invention, and it is possible to retain the proliferation ability in a normal storage state.

The aggregated cancer cell mass of the present invention is an aggregated product formed by unicellularizing a cancer tissue-derived cell mass or a cancer tissue obtained from an individual and causing the mutual aggregation of three or more cells as a whole among the single cells; or causing the mutual aggregation of 3 or more cells as a whole among some cell populations that have not been separated completely into individual cells; or causing the aggregation of 3 or more cells as a whole between the individual cells and the some cells that have not been completely separated; or a cultured product thereof, and the aggregated product and the cultured product can retain proliferation ability in vitro.

Here, the expression of “unicellularizing a cancer tissue-derived cell mass or a cancer tissue obtained from an individual” means that a separation treatment is applied until at least a part of the cancer tissue-derived cell mass or the obtained cancer tissue is allowed to be separated in vitro so that in single cells are contained to some extent. Thus, typically after such a treatment, the expression of “unicellularizing” as used herein corresponds to even in a case where some cells separated into single cells are present and some cells not separated into individual cells are present in a mixed state. At this time, those that are mixed in a state not being separated into individual cells include a cell population with up to 10 cells, and preferably a cell population with 2 or 3 cells.

The expression of “aggregation of 3 or more cells” refers to a state including multiple cells of at least 3 wherein individual cells obtained by unicellularizing a cancer tissue obtained from a cancer that occurs in vivo or from a cancer tissue-derived cell mass that has been found by the present inventor are mutually gathered; or some cell populations that have not been separated into individual cells are mutually gathered; or combinations thereof are mutually gathered.

If the cancer tissue-derived cell mass or the cancer tissue obtained from the cancer that occurs in vivo is subjected to a unicellularization treatment, it includes, but does not limited to, an enzyme treatment of the cancer tissue obtained from an individual.

The enzyme treatment may be a treatment using typically one kind selected from trypsin, dyspase, and optionally collagenase, papain, hyaluronidase, C. histolyticum neutral protease, thermolysin, and dispase or a combination of two or more enzymes thereof. The enzyme treatment conditions may be such that the treatment is carried out in a buffered isotonic salt solution (for example, PBS or Hank's balanced salt solution) having a physiologically acceptable pH of about 6 to 8, and preferably of about 7.2 to 7.6, at for example about 20 to 40° C., and preferably at about 25 to 39° C., for a sufficient time to degrade the connection tissue, for example, about 1 to 180 minutes, and preferably 30 to 150 minutes, at a concentration sufficient for such a purpose, for example, about 0.0001 to 5% w/v, and preferably about 0.001% to 0.5% w/v.

This enzyme treatment may be, but is not limited to, typically a single treatment with trypsin or dyspase.

After the unicellularization treatment, the resulting cells include individually separated cells as well as cells that have not been completely separated into individual cells.

Such cells may be aggregated as they are, but they may be treated with the addition of, for example, an agent to promote the cell aggregation or an agent to suppress the cell death. Examples of the agents include enzyme inhibitors associated with the cell death, such as ROCK inhibitors and caspase inhibitors.

ROCK refers to Rho-associated coiled-coil kinase (ROCK: GenBank accession number: NM_{—}005406), is one of the main effector molecules of Rho GTPase, and is known to control various physiological phenomena (also referred to as Rho-binding kinase). Examples of the ROCK inhibitor include Y27632, and in addition, Fasudil (HA1077), H-1152, Wf-536 (all available from Wako Pure Chemical Industries, Ltd.), and derivatives thereof, and antisense nucleic acids against ROCK, and RNA interference inducing nucleic acids, and vectors containing these nucleic acids.

The treated product that is separated into a population including single cells or 10 or less cells by an enzyme treatment including a trypsin treatment (it is, but is not limited to, a treatment with 0.25% trypsin-EDTA at 37° C. for 5 minutes) is seeded in a 96-well culture plate at a low density (for example, 500 cells/0.32 cm², medium volume: about 0.15 ml) prior to aggregation. The ROCK inhibitor may be added in a concentration of about 1 to 100 μM, and preferably about 10 μM, to a maintenance culture solution immediately or several days after culturing.

Such an aggregated product can be cultured in vitro. The culture time may not be particularly limited as long as the aggregated product is present in the culture medium even for a little time. Such a cultured product often exhibits a substantially spherical shape or a spheroidal shape by culturing the cultured product for a fixed period of time of preferably 3 hours or more. The cultured product herein also includes not only a cultured product having a substantially spherical shape or spheroidal shape after the fixed period of time but also an irregular cultured product before reaching such a shape. Further, the cultured product as used herein includes an irregular shape obtained by further dividing the cultured product having a substantially spherical shape or spheroidal shape and a cultured product having a substantially spherical shape or spheroidal shape obtained by further culture.

The expression that the aggregated cancer cell mass of the present invention “can retain the proliferation ability” in vitro means that the proliferation ability can be retained for a period of time of at least 10 days, preferably 13 days or more, and further preferably 30 days or more, under cell culture conditions of a temperature of 37° C. in 5% CO₂ incubator.

Even when the culture of such an aggregated cancer cell mass is continued as it is, it is possible to retain the proliferation ability for a period of time of 10 days or more, preferably 13 days or more, and further preferably 30 days or more. Further, by carrying out mechanical division at regular intervals during culturing or carrying out the unicellularization treatment and aggregation, the proliferation ability may be retained substantially indefinitely.

Here, the culture medium for the culture of the aggregated cancer cell mass of the present invention is the same as the culture medium for the culture of the cancer tissue-derived cell mass.

The aggregated cancer cell mass of the present invention can be cultured in the culture medium and under such culture conditions. Further, in the culture of the aggregated cancer cell mass, there may be a case where coculture with other cells is preferable or a case where additional special supplements such as hormones may be required, depending on the individual nature.

Specifically, the coculture may be carried out together with feeder cells. Stromal cells such as embryonic fibroblasts and the like can be used as the feeder cells. The feeder cells are not specifically limited, but NIH3T3 or the like is preferred.

Alternatively, in the case of certain kinds of breast cancer, uterine cancer, and prostate cancer, culture is preferably carried out in the presence of a hormone in the same manner as in the cancer tissue-derived cell mass. Specifically, the hormone includes estrogen for breast cancer, progesterone for uterine cancer, testosterone for prostate cancer and the like. However, without being limited to these hormones, it is possible to adjust the culture conditions conveniently by adding various hormones. Further, there may be a possibility to know the hormone-dependency of a cancer derived from a patient and predict the effectiveness of anti-hormonal drug therapy by examining how the behavior of the aggregated cancer cell mass after the culture, for example, the state of life and death as well as the state of proliferation, is changed by the presence of such hormones.

The aggregated cancer cell mass of the present invention may also be cultured by floating cultivation in the same manner as in the cancer tissue-derived cell mass.

The cancer cells constituting the cancer tissue-derived cell mass are at least 3, preferably 8 or more, more preferably 10 or more, and further more preferably 20 or more, and the upper limit is not particularly limited in the number. When the aggregated cancer cell mass of the present invention is a separated product, the number of the cells is preferably 1000 or less, and more preferably about 500 or less. If the cancer cell mass is a cultured product after culturing the separated product, it is possible to increase the number of cells by culture. However, even in the case of a cultured product, the number of cells is preferably 10,000 or less, and more preferably 5000 or less.

The size of the aggregated cancer cell mass of the present invention is not limited, and includes those of irregular shapes having a particle diameter or a volume average particle diameter of about 8 μm to 10 μm, and also includes those having a particle diameter of 1 mm or more largely grown after the culture. The size is preferably 40 μm to 1000 μm in diameter, more preferably 40 μm to 250 μm in diameter, and further more preferably 80 μm to 200 μm in diameter.

The aggregated cancer cell mass of the present invention often has one or more arrangements particularly selected from the group consisting of, but not particularly limited to, palisade arrangement, sheet arrangement, multilayer arrangement, and syncytial arrangement.

The aggregated cancer cell mass of the present invention may be prepared typically by a method including the steps of unicellularizing a cancer tissue extirpated from a living body; and allowing cells among the unicellularized cells to be mutually aggregated to 3 or more cells.

Further, the aggregated cancer cell mass of the present invention may be prepared by a method including, but not limited to, the step of culturing the aggregated component for 3 or more hours.

First, when the aggregated cancer cell mass of the present invention is obtained from the cancer tissue-derived cell mass, the aggregated cancer cell mass itself is subject to an enzymatic treatment, and the cancer tissue itself extirpated from a living body is also subjected to an enzymatic treatment to form unicellularized cells, while fragmentation is preferably carried out before the enzymatic treatment. The cancer tissue can be maintained in a culture medium for animal cell culture before fragmentation. Examples of the culture medium for animal cell culture include, but are not particularly limited to, Dulbecco's MEM (DMEM F12, etc.), Eagle's MEM, RPMI, Ham's F12, alpha MEM, and Iscove's modified Dulbecco's medium. In this case, floating culture is preferably carried out in a culture vessel which is non-adhesive to cells.

It is also preferable to wash the cancer tissue in advance for fragmentation. Such washing can be carried out by using, but not limited to, buffer solutions such as an acetic acid buffer solution (acetic acid+sodium acetate), a phosphoric acid buffer solution (phosphoric acid+sodium phosphate), a citric acid buffer solution (citric acid+sodium citrate), a boric acid buffer solution, a tartaric acid buffer solution, a Tris buffer solution, and a phosphate-buffered saline. In the present invention, the washing of the tissue can be carried out particularly preferably in HBSS. As for the number of times of the washing, once to three times are suitable.

The fragmentation can be carried out by dividing the tissue after washing, with use of a knife, scissors, a cutter (manual operation, automatic operation) or the like. The size and shape after fragmentation are not particularly limited, but the fragmentation may be carried out at random. The tissue is preferably fragmented to a uniform size of preferably 1 mm to 5 mm square, and more preferably 1 mm to 2 mm square.

The fragmented product obtained in this way is then subjected to an enzymatic treatment. Such an enzymatic treatment may be a treatment using mainly trypsin as described above. The conditions therefor may be as follows: at 20 to 45° C. for several minutes to several hours.

The cells among the unicellularized cells obtained in this way are allowed to mutually aggregate to 3 or more cells. It is possible to preferably add a ROCK inhibitor to the unicellularized cells quickly before the aggregation.

Here, the aggregate containing 3 or more cells, obtained by the aggregation, is the aggregated cancer cell mass of the present invention and has a certain range of sizes. The certain range of sizes includes those with a small volume average particle diameter of about 8 μm to 10 μm. When the cell mass is an almost sphere shape, it has a diameter of 20 μm or more and 500 μm or less, preferably 30 μm or more and 400 μm or less, and more preferably 40 μm or more and 250 μm or less. When the cell mass is in a spheroidal shape, it has a long diameter of 20 μm or more and 500 μm or less, preferably 30 μm or more and 400 μm or less, and more preferably 40 μm or more and 250 μm or less. When the cell mass is in an irregular shape, it has a volume average particle diameter of 20 μm or more and 500 μm or less, preferably 30 μm or more and 400 μm or less, and more preferably 40 μm or more and 250 μm or less. The measurement of the volume average particle diameter can be carried out by evaluating the particle diameter distribution and the particle shape using a phase contrast microscope attached with a CCD camera (IX 70; manufactured by Olympus Corporation).

Both the aggregated product and its cultured product, which are obtained in this way, are the aggregated cancer cell mass of the present invention. The cultured product may be those in which the separated product as a component after selection and collection has been present in a culture medium for a short time, or those which are in the shape of a substantially sphere shape or a substantially spheroidal shape after culture for a period of time, for example, at least 3 hours, preferably 10 hours or more and up to 36 hours, and more preferably 24 to 36 hours. The culture time may be over 36 hours, several days, 10 days or more, 13 days or more, or 30 days or more.

The culture may be carried out in a culture medium for a long time without any mechanical division, but the proliferation ability can also be retained for a substantially infinite time period by mechanical division periodically on the way of culture.

Further, the aggregated cancer cell mass of the present invention, even if it includes, for example, 10 or less cancer tissue-derived cell masses (equivalent to 1000 or less cells) with a diameter of 100 μm, has a high engraftment rate in the transplantation in different species of animal. Therefore, the aggregated cancer cell mass of the present invention is useful in the simple and easy production of a cancer model animal including a mouse, and makes it possible to examine a cancer tissue more strictly, evaluate drug sensitivity, or evaluate therapeutic embodiments including a radiation therapy.

The aggregated cancer cell mass of the present invention can be cryopreserved and its proliferation ability can be retained under normal storage conditions.

The cancer tissue-derived cell mass or the aggregated cancer cell mass of the present invention thus obtained shows an in vitro behavior similar to a cancer tissue in a living body and can be stably cultured while retaining its proliferation ability.

Therefore, the cell mass is useful, for example, in identification of the kind of existing drugs to which the tumor that is derived from a cancer tissue obtained is susceptible, or in confirmation of the presence or absence of radiosensitivity in each patient individually. The drug sensitivity or the radiosensitivity can be determined by, but not limited to, any known methods.

Moreover, by culturing the cancer tissue-derived cell mass or the aggregated cancer cell mass and evaluating the cultured cancer tissue-derived cell mass or the aggregated cancer cell mass, if a relationship between genes and drugs or radial rays is known, it is possible to predict in advance the drug sensitivity only by the genetic testing before the drug administration or predict in advance the radiosensitivity. By using the cancer tissue-derived cell mass or the aggregated cancer cell mass, or its culture method of the present invention, such a prediction from a very small amount of specimens becomes possible with very high efficiency so that the burden on patients is reduced and the easy operation becomes possible. In addition, it is also possible to elucidate the unknown relationship between such genes and drug sensitivity or radiosensitivity. That is, although molecular target drugs have been clinically applied as an anti-tumor agent, the need to test in advance the sensitivity and select patients who are susceptible to the drugs has been increased from the viewpoints of side effects and healthcare economics. Since target molecules and intracellular signaling of the molecular target drugs are known, there is a case that can determine the effectiveness of the drug by detecting mutations in the target genes molecular-biologically.

Such genes may not be particularly limited, and may also be genes peculiar to a broad range of various cancers as well as may reflect the trait and metabolism of animals including humans. In particular, a KRAS gene or a BRAF gene is typically exemplified as a gene whose relationship with a drug is known. Of these, it has been elucidated that there is a possibility where the mutations of KRAS or BRAF that is an oncogene can be used to predict the effect of cetuximab that is an antibody pharmaceutical targeted to epidermal growth factor receptor (EGFR) for colorectal cancer. In patients with the mutations, the effect of cetuximab might be insufficient. However, there is a technical limitation in cases or the like without any surgical indications because a judgment must be made from a limited amount of biopsy materials. Particularly, in the case of a cancer tissue with less cancer cell components, it is very difficult to detect abnormalities. The culture method of the present invention has a feature in that a purified cancer cell mass can be prepared and the cell mass can be further expanded. The accurate analysis of KRAS or BRAF genes becomes possible by culturing a very small amount of specimens so that the cancer cells are purified/amplified. Alternatively, gene analysis may be applied to the detection of polymorphisms, such as UGT1A1 genetic polymorphisms. Since this gene is also known to cause low or little sensitivity to anticancer drugs due to its polymorphism, it is possible to avoid the administration of a drug that induces side-effects only by obtaining such information in advance. Such an evaluation may be, for example, to detect the presence or absence of a mutation of the gene. Here, the mutation includes all the diversity, such as deficiency, in addition to base changes. Detection of mutations in the gene may be carried out even by any of known methods, such as direct sequencing of the base included in the gene or evaluation of the restriction enzyme cleavage site.

The step of evaluating the gene may be to detect the gene expression level. Measurement of the gene expression level may be carried out by detecting the expression or the expression level of mRNA that is a genetic transcription product, or similarly by detecting the presence or the presence amount of a protein that is a translation product of the gene or a fragment of the protein. The transcription product of the gene can be detected or measured by known methods for specific detection of the expression of specific genes, such as a Northern blot method, an RT-PCR method, an in situ hybridization method, and a DNA microarray method.

In addition, as for a method for evaluating the gene, the evaluation may also be done by carrying out the culture in the hypoxic state and in the normal oxygen state, and comparing the expression levels of the gene during the culture in the hypoxic state and in the normal oxygen state. Examples of the gene suitable for such an evaluation method include, but are not limited to, VEGF genes. The information obtained from the VEGF gene is related to clinical applications to therapeutic agents such as angiogenesis inhibitors for colorectal cancer. That is, for example, bevacizumab is a humanized monoclonal antibody against vascular endothelial growth factor (VEGF). The VEGF promotes cell division of vascular endothelial cells, and its expression is enhanced in various cancer cells. It is considered that the VEGF promotes angiogenesis to increase the supply of nutrients or oxygen, which is involved in the proliferation and metastasis of cancer cells. Bevacizumab binds specifically to VEGF to inhibit its biological activity so that an anti-cancer activity is exerted. Currently, there is no effective test to predict the drug sensitivity of angiogenesis inhibitors. It is important to evaluate the ability to produce VEGF in tumors, but histopathological analysis of VEGF in the past study does not reflect therapeutic effects. Because the internal environment of tumors is extremely ununiform and thus it is difficult to identify the site where angiogenesis actively occurs, evaluation of the VEGF production as a whole tumor may not necessarily lead to the prediction of the sensitivity. A cancer tissue is known to be hypoxic, and the hypoxia is the strongest VEGF induction factor. In the present invention, it is possible to evaluate the “potential” of cancer cells by changing the culture conditions.

It is possible to store the cancer tissue-derived cell mass or the aggregated cancer cell mass, and cryopreservation is preferably used. The cryopreservation is particularly preferably a method where the cancer tissue-derived cell mass is unicellularized and then aggregation of the unicellularized cells is promoted or the cell death is suppressed. In this manner, the cells may be kept in a good state of storage. Here, even if the unicellularization treatment is carried out, all the cells do not become single cells and the cells not completely separated into individual cells are included. Even in the case of single cells, unicellularized cells are also collected by causing aggregation or adding a drug to suppress the cell death, a favorable state of storage is kept. Here, the unicellularization treatment is a treatment using one kind selected from the group consisting of trypsin, dyspase, and optionally collagenase, papain, hyaluronidase, C. histolyticum neutral protease, thermolysin, and dispase, or a combination of two or more enzymes thereof. Here, examples of the drug to promote the cell aggregation or suppress the cell death include enzyme inhibitors associated with the cell death, such as ROCK inhibitors and caspase inhibitors. In this way, the unicellularized cells are also collected so that a favorable state of storage is kept. In addition, such storage may be kept by a vitrification method.

Possible storage of the cancer tissue-derived cell mass or the aggregated cancer cell mass means that the cell mass can be stored in a state associated with the genetic information of the cancer tissue-derived cell mass or the aggregated cancer cell mass, and such genetic information can be utilized appropriately as needed. The genetic information as used herein may be the information of mutations or expression level differences, similarly to the information of the gene elucidated by the gene evaluation.

In addition, the cancer tissue-derived cell mass or the aggregated cancer cell mass can be stored in a state associated with the clinical information derived from a patient, and such clinical information can be utilized appropriately as needed. The clinical information derived from a patient refers to all clinical information related to general conditions of patients, conditions of local part, sensitivity to drugs, presence or absence of recurrence, survival situation, and the like.

Moreover, it is also possible to store the cancer tissue-derived cell mass or the aggregated cancer cell mass in a state associated with the information of culture conditions for the cancer tissue-derived cell mass or the aggregated cancer cell mass. The information of culture conditions includes, but is not limited to, the presence or absence of hormone dependency and the need of feeder cells, and may further include all information observed during the culturing. Such information, even if constructed in vitro, may highly reflect the in vivo state accurately, and its clinical applications are possible.

In the present invention, examples of a method for measuring the growth rate or survival rate of the cancer tissue-derived cell mass or the aggregated cancer cell mass include a method of observing visually the number of viable cells together with a control example; a method of analyzing images after taking the images with a CCD camera; and a method of measuring colorimetrically an amount of a protein contained in each cell by staining the protein with a protein-binding dye (for example, sulforhodamine B); and a method of measuring an SD (Succinyl dehidrogenase) activity, an MTT activity or an MTS activity.

### INDUSTRIAL APPLICABILITY

The cancer tissue-derived cell mass or the aggregated cancer cell mass of the present invention can be used in vitro for a wide range of applications. In addition, the cell mass can be proliferated by culturing, enabling to proliferate a cancer cell in vitro from a very small amount of specimens. Moreover, the cancer tissue-derived cell mass or the aggregated cancer cell mass of the present invention can be stored, in particular cryopreserved. In addition, it is also possible to carry out an evaluation of genes or an evaluation of culture conditions, and such an evaluation is useful in finding a precise therapeutic method suitable for an individual, based on the information of the evaluation results. That is, the cell mass of the present invention can bring about a dramatic improvement in anti-cancer drugs or radiotherapies that are currently used generally as a trial and error method or a cocktail therapy. Before carrying out such a therapy, relevant information is collected in advance from the cancer tissue-derived cell mass or the aggregated cancer cell mass derived from each patient, and it becomes possible to apply only an effective therapeutic method to a patient. Moreover, since the cancer tissue-derived cell mass or the aggregated cancer cell mass of the present invention may be in such a size that the original cells can be collected with an injection needle or can be cultured, it is also possible to obtain the cell mass from a patient before a surgical operation, as well as to predict an effect of an anti-cancer drug or a radiotherapy with minimal burdens on patients.

### EXAMPLES

Hereinafter, the present invention will be specifically described by way of examples, but the invention is not limited to these examples. In addition, parts and percentages in each example are all based on a weight basis. The culture conditions shown below are, unless otherwise indicated, under conditions of 37° C. in 5% CO₂ incubator. The centrifugal conditions are, unless otherwise specifically stated, 4° C., 1000 rpm, and 5 minutes.

### Example 1

### Preparation of Cancer Tissue-Derived Cell Mass from Human Colorectal Cancer-Transplanted Mice

Human colorectal cancer-transplanted mice were produced by a xenograft procedure as shown below.

At first a surgical resected specimen of a human tumor (colon cancer) is cut into small pieces (each about 2 mm cube) under aseptic conditions. Then, a small incision of about 5 mm was made at the back of mice (nude mice, preferably NOD/SCID mice) with a severe immunodeficiency, and a subcutaneous tissue is peeled from the animal. A tumor graft which has been prepared is subcutaneously inserted, and wound closure is performed with a skin suture clip. Some of the xenografts are observed as a subcutaneous tumor about 14 days later to three months later.

The produced mice bearing a colon cancer were bred under SPF (specific pathogen free) conditions, and when the tumor reached 1 cm in size, it is removed and collected into a 50 ml-centrifugal tube (IWAKI; 2345-050) containing 20 ml of DMEM (Gibco; 11965-092)+1% Pen Strep (Gibco; 15140-022) (both as a final concentration of 100 units/ml penicillin, 100 μg/mL).

Next, after addition of 20 ml of HBSS (Gibco; 14025-092), tumor was washed by inverting the tube for mixing. Then, 20 ml of a fresh HBSS was added, and these procedures were repeated twice, after which time the tumor tissue was transferred to a 10 cm-cell culture dish (Cell Culture Dish) (IWAKI; 3020-100). The necrotic tissue was removed with a surgical knife on this culture dish.

The tumor xenograft from which the necrotic tissue had been removed was transferred to a fresh 10 cm-dish in which 30 ml of HBSS had been added. Then, the tumor graft was fragmented into small pieces (each about 2 mm cube) using a surgical knife.

The fragmented tumor xenograft was transferred to a 50-ml fresh centrifugal tube, centrifuged, the supernatant was discarded, and the residue was washed by inverting the tube for mixing with a 20 ml-HBSS.

The centrifuge and washing were repeated. After that, 20 ml DMEM+1% Pen Strep+0.28 U/ml (final concentration) BLENDZYME 1 (Roche; 11988417001) were added and mixed. This mixture was transferred to a 100 ml-Erlenmeyer flask and treated with LIBERASE BLENDZYME 1 (manufactured by Roche Diagnostics K.K.) in a thermostat bath of 37° C. while rotating it with a stirrer at a low speed for 2 hours.

Then, the enzymatic treatment product was collected into a 50 ml-centrifugal tube, centrifuged, and the supernatant was discarded, after which time 20 ml of HBSS was added and mixed. The mixture was passed through a stainless mesh (500 μm), and the components that passed through the filter were collected into a 50 ml-centrifugal tube, and further centrifuged. After discarding the supernatant, 1 mg/m DNase I solution (Roche; 1284932) (10 mg/ml stock 100 μl+PBS 900 μl) was added to the residue for mixing, and the mixture was allowed to stand at 4° C. for 5 minutes. After that, 20 ml-HBSS was further added, mixed, centrifuged, and the supernatant was discarded. The residue was mixed with 20 ml HBSS, sieved stepwise in the order of 500→250→100 μm, and then passed through a cell strainer of 40 μm (BD; 352340). The cell strainer was soaked in a 10 cm-tissue culture dish (Tissue Culture Dish) containing 30 ml of HBSS, and shaken slightly to remove single cells, small cell masses of 40 μm or less, and debris. The cell strainer was transferred to another 10 cm-tissue culture dish (Tissue Culture Dish) containing 30 ml of HBSS, and the cell mass that had been trapped in the cell strainer was collected by pipetting.

In addition, the same centrifugal separation as above was repeated several times, and 4 ml StemPro hESC SFM (Gibco; A10007-01)+8 ng/ml bFGF (Invitrogen; 13256-029)+0.1 mM 2-mercaptoethanol (Wako; 137-06862)+1% PenStrep+25 μg/ml Amphotericin B (Wako; 541-01961) were added to the resulting components, and mixed. The mixture was transferred to a 6 cm-non-treated dish (EIKEN CHEMICAL Co., Ltd.; AG2000).

This was cultured in an incubator (MCO-17AIC; manufactured by SANYO Electric Co., Ltd.) at 37° C. and 5% CO₂ for 36 hours.

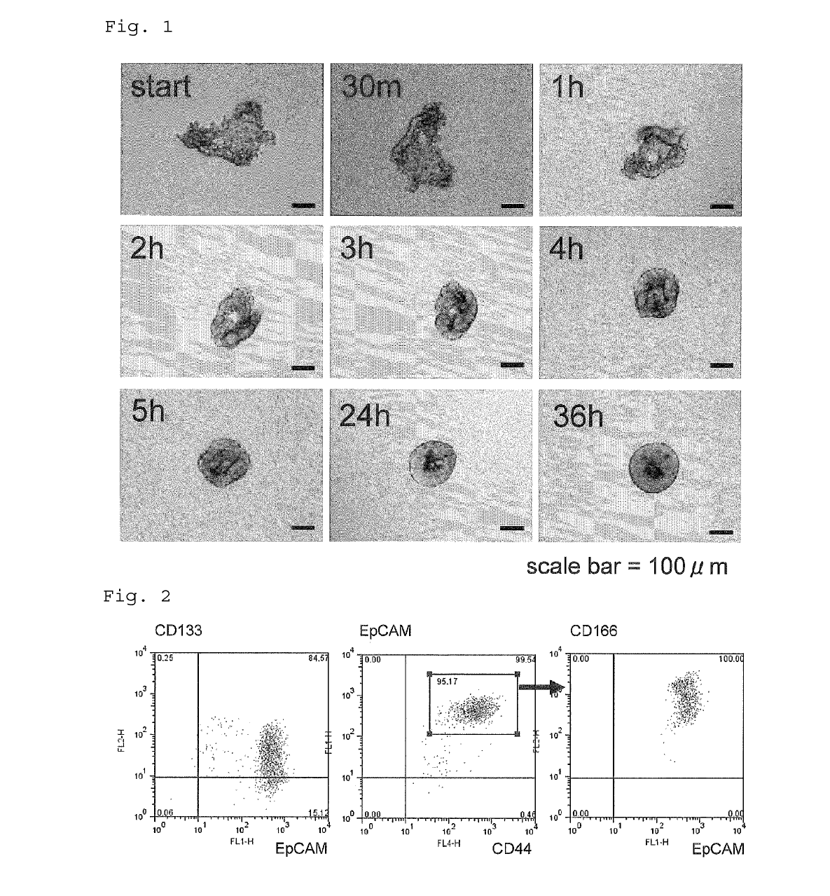

As a result, the cell mass derived from the cancer tissue changed its irregular form into a regular sphere with the lapse of time as shown in FIG. 1, i.e., it became almost a sphere at least 3 to 6 hours later, and a completely regular sphere-shaped cell mass derived from the cancer tissue was obtained after 24 hours.

### Example 2

### Preparation of Cell Mass Derived from Cancer Tissue from Surgical Specimens of Human Colon Cancer

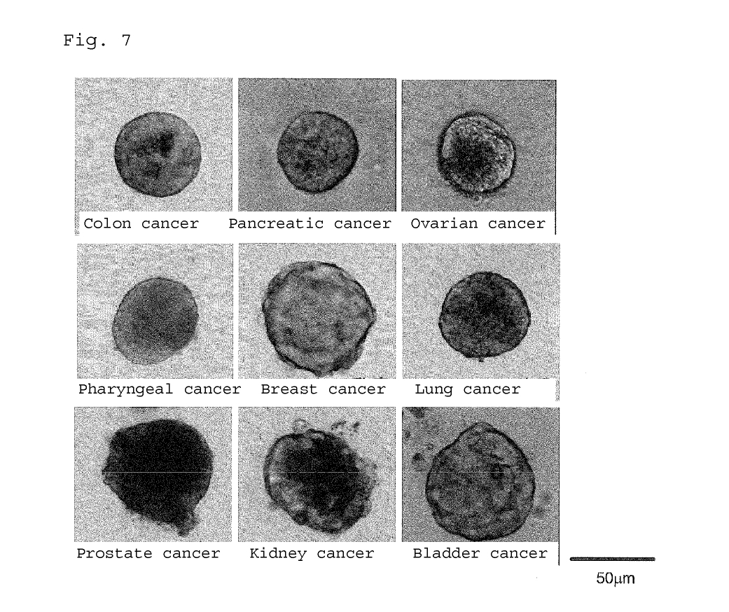

The cell mass derived from the cancer tissue was obtained in the same manner as in Example 1, except that surgical specimens of colon cancer were used. As a result, an almost sphere-shaped cell mass derived from the cancer tissue, similar to one as shown in FIG. 1, was obtained at least 12 hours later as shown in FIG. 7.

### Example 3

### Preparation of Cell Mass Derived from Cancer Tissue from Surgical Specimens of Human Ovarian Cancer

The cell mass derived from the cancer tissue was obtained in the same manner as in Example 2, except that surgical specimens of ovarian cancer were used. As a result, an almost sphere-shaped cell mass derived from the cancer tissue, similar to one as shown in FIG. 1, was obtained at least 12 hours later as shown in FIG. 7.

### Example 4

### Preparation of Cell Mass Derived from Cancer Tissue from Surgical Specimens of Human Pancreatic Cancer

The cell mass derived from the cancer tissue was obtained in the same manner as in Example 2, except that surgical specimens of pancreatic cancer were used. As a result, an almost sphere-shaped cell mass derived from the cancer tissue, similar to one as shown in FIG. 1, was obtained at least 12 hours later as shown in FIG. 7.

### Example 5

### Preparation of Cell Mass Derived from Cancer Tissue from Surgical Specimens of Human Small Cell Lung Cancer

The cell mass derived from the cancer tissue was obtained in the same manner as in Example 2, except that surgical specimens of human small cell lung cancer which is a kind of lung cancers were used. As a result, an almost sphere-shaped cell mass derived from the cancer tissue, similar to one as shown in FIG. 1, was obtained at least 12 hours later as shown in FIG. 7.

### Example 6

### Preparation of Cell Mass Derived from Cancer Tissue from Surgical Specimens of Human Kidney Cancer

The cell mass derived from the cancer tissue was obtained in the same manner as in Example 2, except that surgical specimens of kidney cancer were used. As a result, an almost sphere-shaped cell mass derived from the cancer tissue, similar to one as shown in FIG. 1, was obtained at least 12 hours later as shown in FIG. 7.

### Example 7

### Preparation of Cell Mass Derived from Cancer Tissue from Surgical Specimens of Human Bladder Cancer

The cell mass derived from the cancer tissue was obtained in the same manner as in Example 2, except that surgical specimens of bladder cancer were used. As a result, an almost sphere-shaped cell mass derived from the cancer tissue, similar to one as shown in FIG. 1, was obtained at least 12 hours later as shown in FIG. 7.

### Example 8

### Preparation of Cell Mass Derived from Cancer Tissue from Surgical Specimens of Human Breast Cancer

The cell mass derived from the cancer tissue was obtained in the same manner as in Example 2, except that surgical specimens of breast cancer were used. As a result, an almost sphere-shaped cell mass derived from the cancer tissue, similar to one as shown in FIG. 1, was obtained at least 12 hours later as shown in FIG. 7.

### Example 9

### Preparation of Cell Mass Derived from Cancer Tissue from Surgical Specimens of Human Prostate Cancer

The cell mass derived from the cancer tissue was obtained in the same manner as in Example 2, except that surgical specimens of prostate cancer were used. Dihydrotestosterone (DHT) with a concentration of 10⁻⁸ mol/L was added to a medium, and culture was performed in the same manner as in Example 1. As a result, an almost sphere-shaped cell mass derived from the cancer tissue, similar to one as shown in FIG. 1, was obtained at least 12 hours later as shown in FIG. 7.

### Example 10

### Preparation of Cell Mass Derived from Cancer Tissue from Surgical Specimens of Human Pharyngeal Cancer

The cell mass derived from the cancer tissue was obtained in the same manner as in Example 2, except that surgical specimens of pharyngeal cancer were used. As a result, an almost sphere-shaped cell mass derived from the cancer tissue, similar to one as shown in FIG. 1, was obtained at least 12 hours later as shown in FIG. 7.

### Example 11

### Hormone Sensitivity Test of Cell Mass Derived from Breast Cancer Tissue

An investigation was made on how the state of each of the cell masses derived from the cancer tissues from a plurality of patients with breast cancers was different from each other by the presence or absence of estradiol under the same medium conditions as in Example 8. As a result, as shown in FIG. 8, it has been understood that there are a case where proliferation is promoted by the addition of estradiol and a case that does not respond to estradiol. This was found to be applicable as a sensitivity test in a hormone therapy of a patient from which the cell mass was derived.

### Example 12

### Preparation of Cell Mass Derived from Cancer Tissue from Mouse Pancreatic Islet Cell Tumor

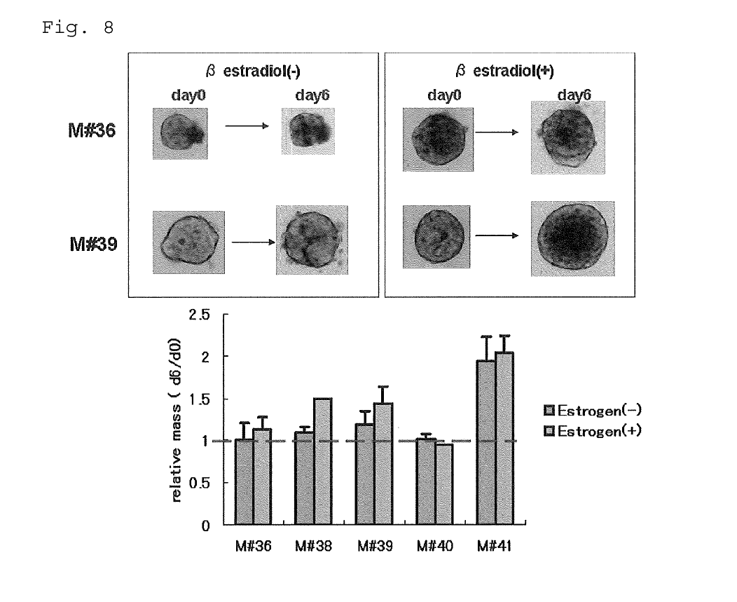

RipTag is a transgenic mouse wherein SV40-T antigen is forcedly expressed under the control of a rat insulin promoter and a tumor occurs in the pancreatic islet. The cell mass derived from the cancer tissue was obtained in the same manner as in Example 2, except that the pancreatic islet tumor in RipTaq mice was used. As a result, an almost sphere-shaped cell mass derived from the cancer tissue, similar to one as shown in FIG. 1, was obtained at least 12 hours later (FIG. 9).

### Example 13

The cell mass derived from the cancer tissue under culture as shown in FIG. 7 obtained in Example 2 was taken out together with 5 ml of the medium 24 hours after culture, centrifuged at 1000 rpm and 4° C., and the supernatant was discarded. The collected cell mass derived from the cancer tissue was suspended in Cell Banker (BLC-1, manufactured by Mitsubishi Chemical Medicine Corporation) and 10 μM of Y27632 (manufactured by Wako Pure Chemical Industries, Ltd.) was further added thereto. The mixture was transferred to a cryopreservation tube (Cryogenic vials 2.0 ml, manufactured by Nalge Nunc Corporation) and preserved in a deep freezer at −80° C.

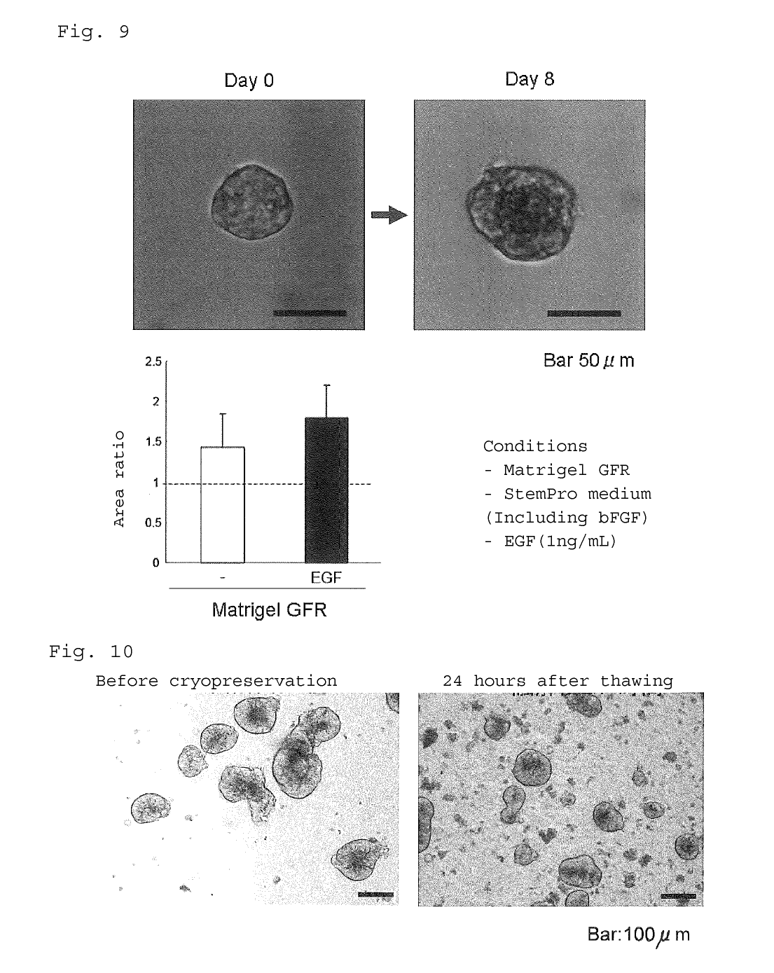

After 7-days preservation, the mixture was rewarmed in a water-bath of 37° C. for a short time. This was suspended in PBS, centrifuged at 1000 rpm and 4° C., and the supernatant was discarded. The resultant precipitate was suspended in StemPro (manufactured by Invitrogen) and cultured. As shown in FIG. 10, the cell state at 24 hours after thawing was excellent.

Furthermore, the survival of the resulting cell mass derived from the cancer tissue was confirmed by transplanting it into NOD-SCID mice as a mass containing approximately 1,000 cells.

### Example 14

### Preparation of Aggregated Cancer Cell Mass from Cancer Tissue-Derived Cell Mass

The following treatment was carried out using the cancer tissue-derived cell mass obtained in the same manner as in Example 2. First, collagen gel (50 μL/well) (Cell Matrix type I-A:5×DMEM:buffer solution for gel reconstruction=7:2:1) was spread in the center of a 24-well plate (untreated dish). The plate was allowed to stand at 37° C. for 30 minutes so that the collagen gel was solidified. The cancer tissue-derived cell masses (100 per well) obtained by the floating culture were collected in a 1.5 mL-tube. The culture was centrifuged for about 5 seconds and the supernatant was removed. The cancer tissue-derived cell mass was suspended in collagenase gel (30 μL/well) and placed (30 μL each) on the gel that had been solidified in advance. After allowing the suspension to stand at 37° C. for 30 minutes for solidification, and StemPro (EGF 50 ng/mL) was added each in amount of 600 μL/well. While exchanging the culture medium once every 2 to 3 days, the cells were cultured for 10 days.

Then, the culture medium was exchanged with DMEM (1 mL/well) (Gibco; 11965-092, including collagenase IV (200 mg/mL)), and the cells were cultured at 37° C. for about 5 hours.

After the incubation, the culture was transferred to a 1.5 mL-Eppendorf tube, and centrifuged (about 5 seconds), the supernatant was removed, the residue was suspended with the addition of 1 ml of PBS, the suspension was centrifuged (Chibitan, about 5 seconds), and the removal of the supernatant was repeated twice. Then, 1 mL of Trypsin/EDTA (0.05%) was added to the residue for suspension, and the suspension was allowed to stand at 37° C. for 8 minutes. The suspending was carried out several times to confirm that a large mass like the cancer tissue-derived cell mass was disappeared. This was transferred to a 15 mL-tube, and suspended after addition of 2 mL of DMEM (Gibco; 11965-092).

Then the suspension was centrifuged (1000 rpm, 5 minutes), and the supernatant was removed. The residue was suspended with 2 mL of StemPro (EGF 50 ng/mL, Y-27632, 10 μM), and transferred to a φ35 mm non-treated dish (Iwaki: 1000-035). This was cultured overnight at 37° C.

After 12 hours, formation of a cancer tissue-derived cell mass of about 40 μm in diameter was confirmed. The culture medium was replaced with StemPro (EGF 50 ng/mL).

As a result, an aggregated cancer cell mass in the form of completely regular sphere shape was obtained 4 days later, as shown in FIG. 11.

### Example 15

### Preparation of Aggregated Cancer Cell Mass from Surgical Specimens of Human Colorectal Cancer

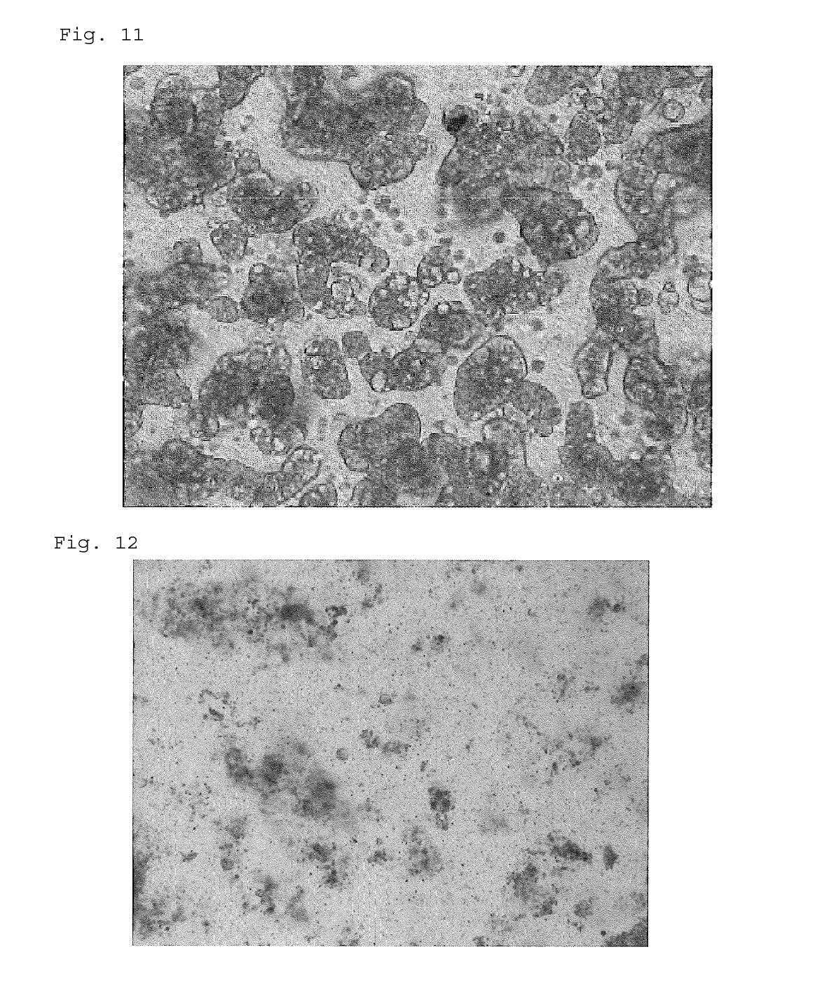

An aggregated cancer cell mass was obtained in the same manner as Example 14, except that surgical specimens of human colorectal cancer were used. As a result, an aggregated cancer cell mass in a substantially sphere shape similar to the cell mass as shown in FIG. 1 was obtained at least 12 hours later as shown in FIG. 12.

### Example 16

Cell storage of a cancer tissue-derived cell mass obtained in the same manner as in Example 2 was carried out. The cancer tissue-derived cell mass was unicellularized by trypsin treatment in the same manner as in Example 14. Cell Banker 1 (Juji Field Inc.) to which Y-27632 had been added was used as a cryopreservation solution.

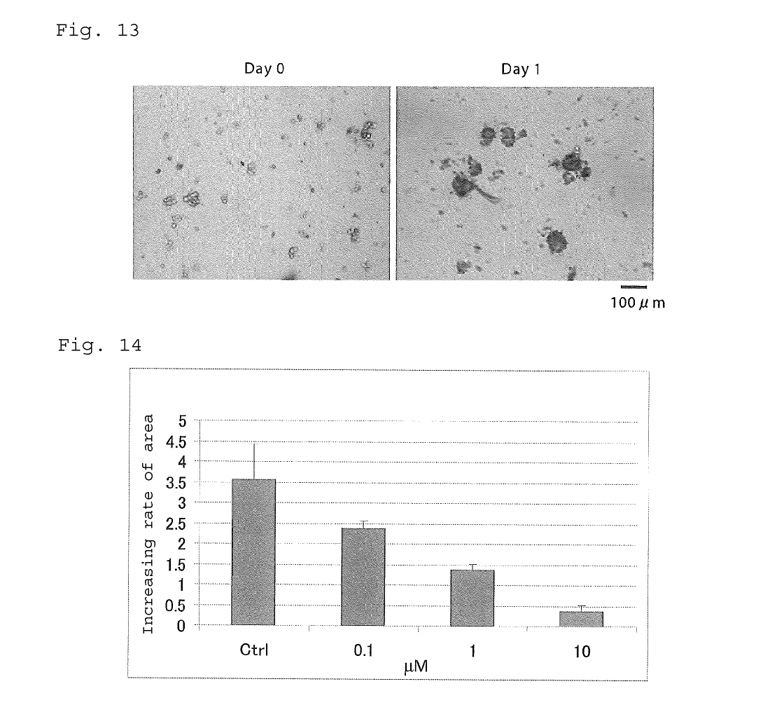

The unicellularized cells which had been cryopreserved for 10 days were then rewarmed under heating in a water bath of 37° C. for a short time. This was suspended in PBS, centrifuged at 1000 rpm, at 4° C. and the supernatant was discarded. The resulting precipitate was suspended in StemPro (manufactured by Invitro), and cultured. As shown in FIG. 13, the state of the cells 24 hours after thawing was favorable, and the cancer tissue-derived cell mass was reconstructed after thawing.

### Comparative Example 1

A sample which had been subjected to the unicellularization treatment according to the method described in the literature (Todaro M. et al., (2007) Colon cancer stem cells dictate tumor growth and resist cell death by production of interleukin-4. Cell Stem Cell 1:389-402) was prepared using surgical specimens of human colorectal cancer. However, in vitro proliferation was not found in CD133 positive cells that had been selected after unicellularization treatment.

Evaluation items in examples and the like were measured as follows.

### <Identification of Surface Antigen>

### <Identification of Surface Antigen>

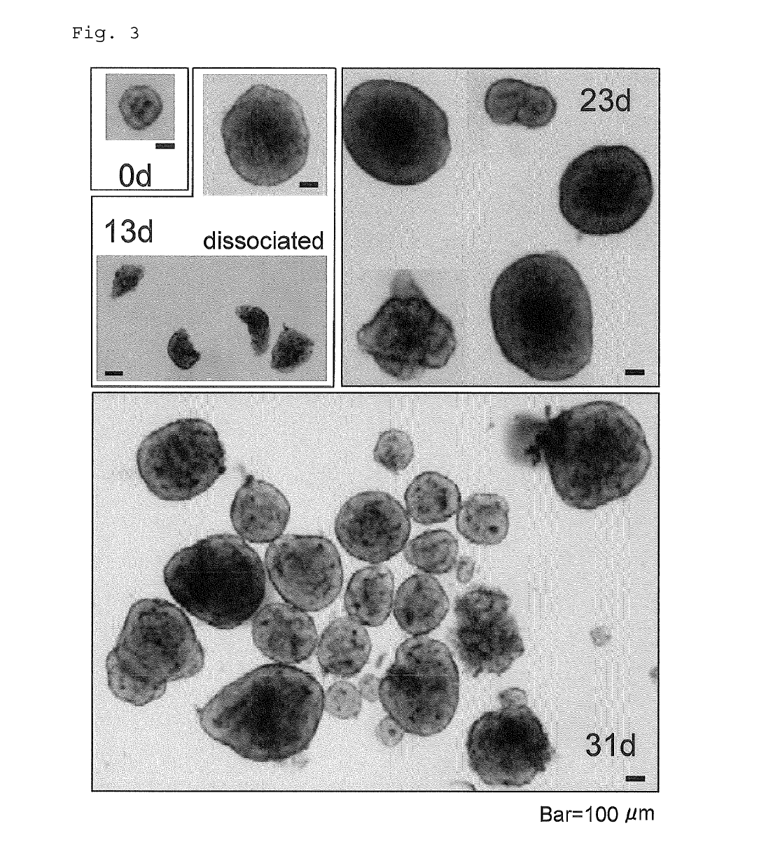

The cell mass from the cancer tissue, obtained in Example 1, was dispersed to single cells using trypsin/EDTA. These cells were reacted with a surface antigen-specific antibody that was labeled with a fluorescence substance, and then analyzed by a flow cytometry. As a result, the existence of cells that expressed a surface antigen uniformly at the same time was recognized as shown in FIG. 2.

### <Confirmation of Basement Membrane-Like Material>

The cell mass derived from the cancer tissue, obtained in Example 1, was cultured for three days in 1 cc of STEMPRO serum-free medium (Gibco) for human ES cells in an incubator under the culture conditions of 37° C. and 5% CO₂. Antigenicity of laminin was observed in the cytoplasm of the cell in or near to the circumference of the cell mass derived from the cancer tissue when this was fixed with formalin, embedded in paraffin, cut into thin slices, and anti-laminin antibody staining (mouse laminin-derived rabbit antibody; manufactured by Sigma-Aldrich Corporation) was performed according to the manufacturer's instructions. As a result, in the cell mass derived from a cancer tissue according to the present invention, it was found that laminin surrounded the circumference of a population of the cancer cells. On the other hand, expression of laminin was not confirmed within 24 hours after treatment of surgical specimens.

### <Detection of Hypoxia>

### Example of Hypoxia Detection Using Pimonidazole

Pimonidazole that is a nitroimidazole compound has a characteristic to form an adduct with proteins or nucleic acids in the absence of oxygen. The hypoxic region of the tissue treated with pimonidazole under hypoxic conditions can be recognized using an antibody that specifically recognizes pimonidazole. When the cancer tissue was separated by about 100 micrometers from a blood vessel, a hypoxic region appears, and a wide range of cell death was observed inside (hypoxic region) the boundary apart from about 100 micrometers from the circumference of even the cell mass derived from the cancer tissue obtained in Example 1.

### <Evaluation of In Vitro Proliferation Ability>

The in vitro proliferation ability of the cell mass derived from a cancer tissue was examined as follows. The cell masses (×10 each) derived from the cancer tissue, obtained in Example 1, were embedded in a collagen gel (CellMatrix type IA (Nitta Gelatin Inc.):5×DMEM (Gibco; 12100-038):buffer solution for gel reconstruction (50 mM NaOH, 260 mM NaHCO3, 200 mM HEPES)=7:2:1), and was cultured in 1 cc of STEMPRO serum-free medium (Gibco) for human ES cells in an incubator under the culture conditions of 37° C. and 5% CO₂. The cell state was observed periodically and the size of the cell was measured with a phase contrast microscope (magnification 40 times) equipped with a CCD camera. As a result, without mechanical division, the proliferation ability could be retained for at least 13 days as shown in FIG. 3. Moreover, it was confirmed that the proliferation ability could be retained for further at least 13 days when mechanical division was performed on day 13. In addition, the mechanical division of the cell mass was performed by dividing the cell mass with a diameter of 500 micrometers derived from the cancer tissue into four with an ophthalmic pointed knife.

### <Confirmation of Cell Count>

A 100 to 250 μm-sized cell mass derived from a cancer tissue was treated with trypsin 0.25% and EDTA 2.6 mM for three minutes in the same manner as in Example 1, and mechanically degraded by pipetting approximately 30 times. This was diluted and subdivided into a 96-well culture plate so that one cell can be placed in one well. The cell count constituting a cell mass that was non-single celled was counted and recorded. Then, culture (under the conditions as above) was performed to record an increase of the cell count of each well, and the culture was observed for 30 days. As a result, it was confirmed that a cell mass could be even grown up if there were three cells.

### <Drug Sensitivity Test>

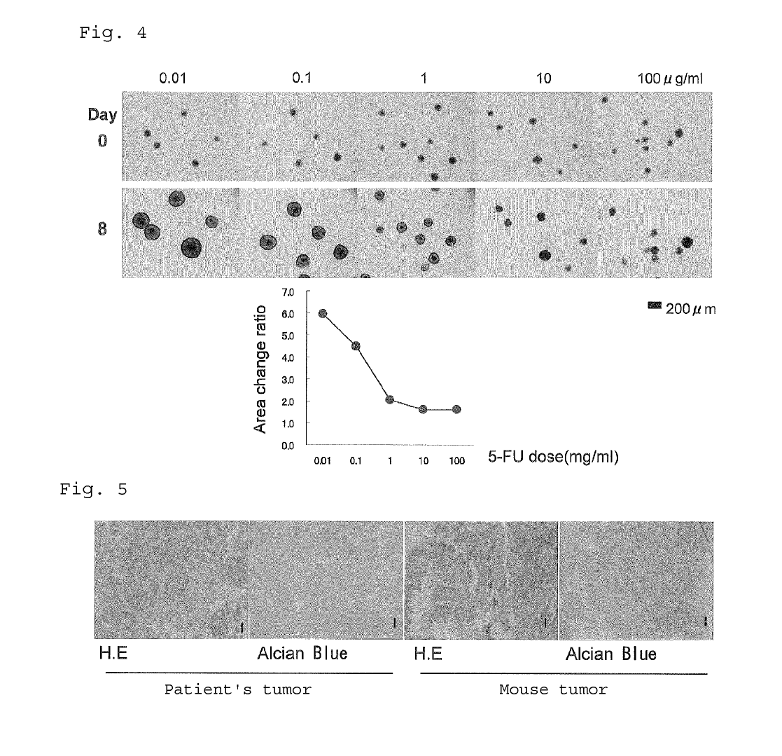

Using 5-FU which is known to inhibit DNA synthesis by binding to a thymidylic acid synthetase involved in the metabolism process necessary for DNA synthesis, a drug sensitivity test on a sample of Example 2 was carried out. The test was carried out by embedding the cell masses (×10 each) derived from the cancer tissue in a collagen gel (CellMatrix type IA (Nitta Gelatin Inc.):5×DMEM (Gibco; 12100-038):buffer solution for gel reconstruction (50 mM NaOH, 260 mM NaHCO3, 200 mM HEPES)=7:2:1), and culturing in 1 cc of STEMPRO serum-free medium (Gibco) for human ES cells in an incubator under the culture conditions of 37° C. and 5% CO₂. In addition, 5-FU was applied at a concentration of 0.01 μg/ml, 0.1 μg/ml, 1 μg/ml, 10 μg/ml, and 100 μg/ml, and the states of the cells on days 0 and 8 after culture were compared for evaluation. The results are shown in FIG. 4. An increasing rate of the area of the cell mass derived from the cancer tissue was expressed relative to 1 of an increasing rate of the area of the cell mass in the culture without application of a drug. In FIG. 4, it was actually demonstrated that proliferation of the cancer cell was concentration-dependently suppressed by 5-FU on day 8 after culture, and the cell mass derived from a cancer tissue according to the present invention was useful in a drug sensitivity test.

### <Transplantation Test in Different Species of Animals>

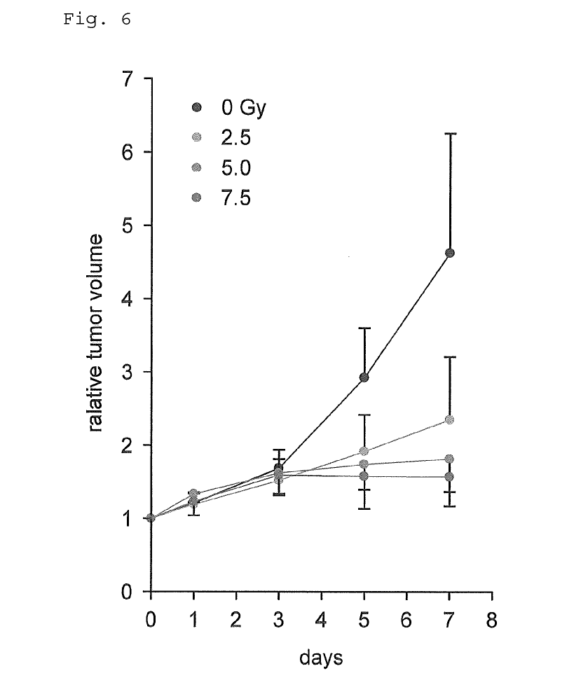

The cell masses (×10) having each a diameter of about 100 μm derived from the cancer tissue, obtained in Example 2 by culture for three days according to the present invention, were suspended in Matrigel (BD Corporation), and the suspension was administered subcutaneously to the back of NOD-SCID mice for transplantation. The evaluation of tumorigenesis was performed by measuring the size of the tumor with the lapse of time. As a result, it was confirmed that a marked tumorigenesis was recognized in an individual of mice which had been transplanted with the cell mass derived from the cancer tissue of Example 2 of the present invention, and the cell mass derived from the cancer tissue according to the present invention has a high tumorigenic ability. When this tissue was analyzed, it was revealed that a similar tissue type was produced in both of the tumor occurred in transplanted mice and the existing tumor in a living body (FIG. 5).

### <Radiation Irradiation Test>

The cell masses derived from the cancer tissue obtained in Example 2 and used in the present invention, having a diameter of about 100 μm, were embedded in a collagen gel (CellMatrix type IA (Nitta Gelatin Inc.):5×DMEM (Gibco; 12100-038):buffer solution for gel reconstruction (50 mM NaOH, 260 mM NaHCO3, 200 mM HEPES)=7:2:1), and inoculated (×10 cell masses each) to 1 cc of STEMPRO serum-free medium (Gibco) for human ES cells in an incubator under the culture conditions of 37° C. and 5% CO₂ and then cultured. This was irradiated by γ-rays emitted from a cobalt isotope as a radiation source, thereby to confirm the state of the cell mass. The results are shown in FIG. 6. In FIG. 6, it was actually demonstrated that proliferation of the cancer cell until the 8^{th} day after culture was suppressed depending on the exposure dose, and the cell mass derived from a cancer tissue according to the present invention was useful in a radiation irradiation test.

### <Drug Sensitivity Test>

Using doxorubicin that is known to exert an antitumor effect by suppressing the biosynthesis of both DNA and RNA as a result of inhibiting the reaction of DNA polymerase, RNA polymerase, and topoisomerase II due to the insertion of doxorubicin between the base pairs of DNA of tumor cells, a drug sensitivity test was carried out by the sample of Example 12. The test was carried out by embedding the aggregated cancer cell mass (×10 each) in collagen gel (CellMatrix type IA (Nitta Gelatin):5×DMEM (Gibco; 12100-038):buffer solution for gel reconstruction (50 mM NaOH, 260 mM NaHCO₃, 200 mM HEPES)=7:2:1) and carrying out culture in 1 cc of a STEMPRO human ES cell serum-free medium (Gibco) under the culture conditions of a temperature of 37° C. in 5% CO₂ incubator. In addition, doxorubicin was applied at a concentration of 0.1 μM, 1 μM, and 10 μM, and the states on day 0 and on day XX were compared for evaluation. The results are shown in FIG. 14. An increasing rate of the area of the aggregated cancer cell mass was relatively expressed when an increasing rate of the area of the cell mass in the culture without application of a drug was determined to be 1. In FIG. 14, it was actually demonstrated that proliferation of the cancer cell on day 8 after culture initiation was concentration-dependently suppressed by doxorubicin and the aggregated cancer cell mass of the present invention is useful in a drug sensitivity test.

### <Detection of Gene Mutation>

DNA was extracted from about 100 cancer tissue-derived cell masses on day 2 after culture initiation prepared in the same manner as in Example 1 and Example 2 (sample 1 and sample 2, respectively) with use of DNeasy Blood and Tissue (Quagen), and its 1/100 amount was amplified by the PCR method. Using this as a template, DNA was sequenced by a direct sequencing method according to a conventional method. As a result, it was found that glycine at position 12 of KRAS in sample 1 is replaced by valine, and aspartic acid at position 593 of BRRAF in sample 2 is replaced by glycine, as shown in FIG. 15. In the patients of these samples, it is expected that cetuximab is not effective.

Because the cancer tissue-derived cell mass is composed of pure cancer cells, it is suitable for the detection of gene mutation in cancer cells. In the case of a usual sample frequently contaminated with normal cells, the relative proportion of cancer cells having a mutation is decreased, and thus the detection sensitivity to the mutation is significantly reduced. Therefore, in a conventional method that has been applied so far, only the cancerous part had to be cut out from tissue sections in a manner such as laser capture microdissection. On the other hand, the detection sensitivity to the cancer tissue-derived cell mass increases dramatically because there is no contamination of normal cells in the cancer tissue-derived cell mass. By using the cancer tissue-derived cell mass, it was actually demonstrated that gene mutations can be detected easily in a short period of time by direct sequencing.

### <Sensitivity Test of Angiogenesis Inhibitor>

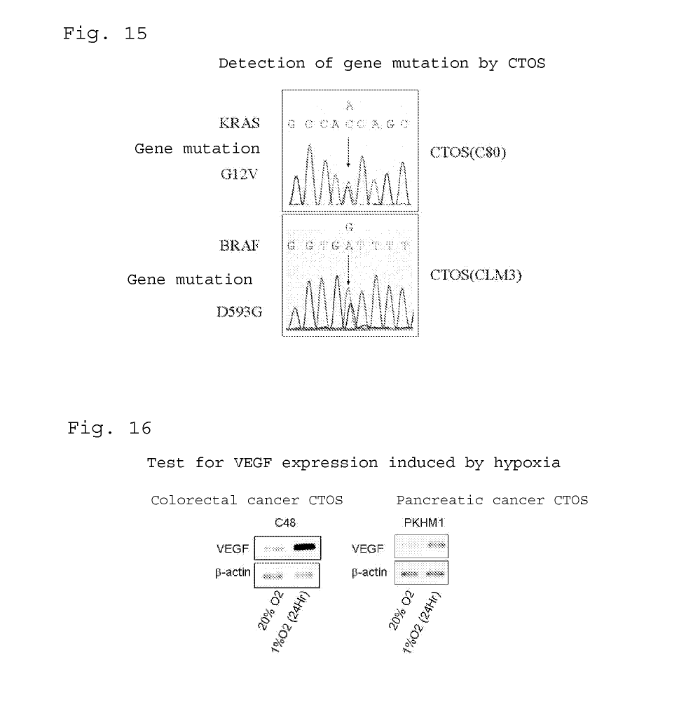

The cancer tissue-derived cell masses prepared in the same manner as in Examples 2 and 4 were compared between when cultured in a floating state for 24 hours using StemPro under a normal oxygen concentration at 37° C. and 5% CO₂ and when cultured with a multi-gas incubator (ASTEC) under a low oxygen concentration of 1% at 37° C. and 5% CO₂. Total mRNA was extracted, and the expression of VEGF genes was detected by the RT-PCR method. As a result, as shown in FIG. 16, the expression of VEGF gene was observed in the cancer tissue-derived cell mass of the present invention under hypoxic conditions, which accurately reflected the in vivo state, so that application possibility of bevacizumab could be confirmed.

1. A method for culturing a cancer tissue-derived cell mass or an aggregated cancer cell mass, comprising culturing the cancer tissue-derived cell mass or an aggregated cancer cell mass in a culture medium obtained by adding a serum replacement to a serum-free basal culture medium, wherein said cancer tissue-derived cell mass or an aggregated cancer cell mass is in the form of composition comprising a plurality of cultured cancer tissue-derived cell masses, wherein each of the plurality of masses comprises a plurality of cancer cells of a separated product that is separated from a cancer tissue obtained from an individual as a mass and wherein each of the cultured cancer tissue-derived cell mass takes almost spherical or ellipsoidal form and can retain a proliferation ability in vitro and wherein the composition does not contain substantially any cells other than cancer cells.

2. The method for culturing according to claim 1, wherein the culture medium obtained by adding a serum replacement to a serum-free basal culture medium is STEMPRO (registered trade mark).

3. The method for culturing according to claim 1, wherein the cancer tissue-derived cell mass or the aggregated cancer cell mass is derived from colorectal cancer, ovarian cancer, breast cancer, lung cancer, prostate cancer, uterine cancer, kidney cancer, bladder cancer, pharyngeal cancer, or pancreatic cancer.

4. The method for culturing according to claim 1, wherein the culture is further carried out with the addition of a hormone to the culture medium.

5. The method for culturing according to claim 4, wherein the cancer tissue-derived cell mass or the aggregated cancer cell mass is derived from a cancer selected from the group consisting of breast cancer, uterine cancer, and prostate cancer, and the hormone is at least a hormone selected from the group consisting of estrogen, progesterone, and testosterone.

6. The method for culturing according to claim 1, wherein the cancer tissue-derived cell mass or the aggregated cancer cell mass is divided every fixed period of time during the culture.

7. A method for evaluating hormone dependency of a cancer tissue-derived cell mass or an aggregated cancer cell mass, comprising the steps of culturing the cancer tissue-derived cell mass or the aggregated cancer cell mass in the presence or absence of a hormone, wherein said cancer tissue-derived cell mass or an aggregated cancer cell mass is in the form of composition comprising a plurality of cultured cancer tissue-derived cell masses, wherein each of the plurality of masses comprises a plurality of cancer cells of a separated product that is separated from a cancer tissue obtained from an individual as a mass and wherein each of the cultured cancer tissue-derived cell mass takes almost spherical or ellipsoidal form and can retain a proliferation ability in vitro and wherein the composition does not contain substantially any cells other than cancer cells; and comparing the state of the cancer tissue-derived cell mass or the aggregated cancer cell mass by the presence or absence of the hormone after the culture.

8. The method for evaluating hormone dependency according to claim 7, wherein the cancer tissue-derived cell mass or the aggregated cancer cell mass is derived from a cancer selected from the group consisting of breast cancer, uterine cancer, and prostate cancer, and the hormone is at least a hormone selected from the group consisting of estrogen, progesterone, and testosterone.

9. The method for evaluating hormone dependency according to claim 7, wherein the comparison step is to compare the state of proliferation or the state of life and death of the cancer tissue-derived cell mass or the aggregated cancer cell mass.

10. A method for evaluating a cancer tissue-derived cell mass or an aggregated cancer cell mass, comprising the steps of culturing the cancer tissue-derived cell mass or the aggregated cancer cell mass wherein said cancer tissue-derived cell mass or an aggregated cancer cell mass is in the form of composition comprising a plurality of cultured cancer tissue-derived cell masses, wherein each of the plurality of masses comprises a plurality of cancer cells of a separated product that is separated from a cancer tissue obtained from an individual as a mass and wherein each of the cultured cancer tissue-derived cell mass takes almost spherical or ellipsoidal form and can retain a proliferation ability in vitro and wherein the composition does not contain substantially any cells other than cancer cells; and evaluating the gene of the cultured cancer tissue-derived cell mass or the cultured aggregated cancer cell mass.

11. The method for evaluating a cancer tissue-derived cell mass or an aggregated cancer cell mass according to claim 10, wherein the gene is a KRAS gene or a BRAF gene, and the evaluation is to detect the presence or absence of a gene mutation.

12. The method for evaluating a cancer tissue-derived cell mass or an aggregated cancer cell mass according to claim 10, wherein the step of evaluating the gene is to detect the expression level of the gene.

13. The method for evaluating a cancer tissue-derived cell mass or an aggregated cancer cell mass according to claim 12, wherein the culture is carried out in a hypoxic state or in a normal oxygen state, and the step of evaluating the gene is to compare the expression level of the gene in the culture in the hypoxic state or in the normal oxygen state.

14. The method for evaluating according to claim 12, wherein the gene is a VEGF gene.

15. A method for storing a cancer tissue-derived cell mass or an aggregated cancer cell mass by a freezing method, wherein said cancer tissue-derived cell mass or an aggregated cancer cell mass is in a form of composition comprising a plurality of cultured cancer tissue-derived cell masses, wherein each of the plurality of masses comprises a plurality of cancer cells of a separated product that is separated from a cancer tissue obtained from an individual as a mass and wherein each of the cultured cancer tissue-derived cell mass takes almost spherical or ellipsoidal form and can retain a proliferation ability in vitro and wherein the composition does not contain substantially any cells other than cancer cells.

16. The method for storing according to claim 15, wherein the method comprises a unicellularization treatment of a cancer tissue-derived cell mass and a treatment for promoting cell aggregation or a drug treatment for suppressing cell death.

17. The method for storing according to claim 16, wherein the unicellularization treatment is a treatment using one kind selected from the group consisting of trypsin, dyspase, collagenase, papain, hyaluronidase, C. histolyticum neutral protease, thermolysin, and dispase, or a combination of two or more enzymes thereof, and the treatment for promoting cell aggregation or the drug treatment for suppressing cell death is a treatment with a ROCK inhibitor or a caspase inhibitor.

18. The method for storing according to claim 15, which is carried out by a vitrification method.

19. The method for storing according to claim 15, wherein the cancer tissue-derived cell mass or the aggregated cancer cell mass is stored in a state associated with genetic information belonging to the cancer tissue-derived cell mass or the aggregated cancer cell mass.

20. The method for storing according to claim 15, wherein the cancer tissue-derived cell mass or the aggregated cancer cell mass is stored in a state associated with clinical information derived from a patient.

21. The method for storing according to claim 15, wherein the cancer tissue-derived cell mass or the aggregated cancer cell mass is stored in a state associated with information of culture conditions for the cancer tissue-derived cell mass or the aggregated cancer cell mass.

22. The method for storing according to claim 21, wherein the information of culture conditions is the presence or absence of hormone dependency.
